(19) United States
(12) Patent Application Publication
(10) Pub. No.: US 2017/0164924 A1
(43) Pub. Date: Jun. 15, 2017

(54) ULTRASOUND IMAGE DIAGNOSTIC APPARATUS

(71) Applicant: Konica Minolta, Inc., Tokyo (JP)

(72) Inventors: Makiko URABE, Yokohama-shi (JP); Kazuya TAKAGI, Tokyo (JP)

(21) Appl. No.: 15/380,154

(22) Filed: Dec. 15, 2016

(30) Foreign Application Priority Data
Dec. 15, 2015 (JP) 2015-244110
Aug. 25, 2016 (JP) 2016-164296

(51) Int. Cl. A61B 8/08; A61B 8/15

(52) U.S. Cl. CPC A61B 8/08; A61B 8/15; A61B 8/52

(57) ABSTRACT

An ultrasound image diagnostic apparatus includes the following. An image generator generates tomographic image data of an object from generated acoustic ray data. A measuring target acknowledging unit acknowledges a measuring target of the object from the generated tomographic image data is stably drawn for a predetermined amount of time and when it is acknowledged, advances to a measuring performing mode of the measuring target. An initial condition obtaining unit obtains initial condition information for extracting an outline of the measuring target. An outline extractor uses the obtained initial condition information to extract an outline of the measuring target from the generated tomographic image data. A measuring unit obtains diameter information of the measuring target based on the extracted outline and calculates the feature amount of the measuring target from the diameter information.

### CROSS-REFERENCE TO RELATED APPLICATIONS

The entire disclosure of Japanese Patent Application No. 2015-244110 filed on Dec. 15, 2015, and Japanese Patent Application No. 2016-164296 filed on Aug. 25, 2016, including descriptions, claims, drawings and abstracts are incorporated herein by reference in its entirety.

### BACKGROUND OF THE INVENTION

Field of the Invention

The present invention relates to an ultrasound image diagnostic apparatus.

Description of Related Art

With ultrasound diagnosis, movement of the heart or fetus can be obtained as an ultrasound image by simple operation such as placing an ultrasound probe against a surface of the body. Since such ultrasound diagnosis is highly safe, the examination can be repeated. Such ultrasound image diagnostic apparatus which is used for ultrasound diagnosis and which displays the ultrasound image is well known.

The ultrasound image diagnostic apparatus is used to scan and display a tumor as a measuring target of an object and the feature amount of the tumor is measured. The feature amount which is to be the measuring target of the tumor is mainly a depth/width ratio, and tumor diameter. When the tumor is a malignant tumor, such tumor has a characteristic of being hard and hardly becomes flat. Therefore, the depth/width ratio is mainly used in diagnosis to judge whether the tumor is a benign tumor or a malignant tumor. The tumor diameter is used to observe over time the effect of chemotherapy before operation.

Conventionally, the user operates the ultrasound image diagnostic apparatus to specify on the tumor in the ultrasound image a caliper mark in the width diameter parallel to the skin surface of the object, and then a caliper mark in the depth diameter orthogonal to the above. Then, the user uses the width diameter and the depth diameter obtained from the caliper mark to mentally calculate the depth/width ratio of the tumor. Also conventionally, the user specified on the tumor in the ultrasound image the caliper mark in the maximum diameter in the depth, width, diagonal directions, and the user used the diameter information obtained from the caliper mark to mentally calculate the tumor diameter. However, there is known an apparatus in which part of the calculations to obtain the feature amount of the tumor is measured automatically to reduce the burden of the user.

For example, there is known an ultrasound diagnostic apparatus in which the user specifies the maximum width diameter L of the tumor and a depth diameter guide of k·L (k: coefficient for determining benign or malignant tumor set in advance by the user) is displayed (see Japanese Patent Application Laid-Open Publication No. 2003-204962). Also there is known an ultrasound system in which the inverted trough method is used to identify the interest region, the gray level is three-dimensionally plotted to segment the interest region, the maximum region among the segmented region is to be the main affected part, and the ratio of the short axis of the oval which most matches the affected part (tumor) is used to automatically determine the depth/width ratio (see Japanese Patent Application Laid-Open Publication No. 2005-193017).

However, according to the ultrasound diagnostic apparatus described in Japanese Patent Application Laid-Open Publication No. 2003-204962, although it is possible to confirm by sight whether the depth/width ratio exceeded the predetermined reference value, the depth/width ratio cannot be measured without user operation. The user needs to specify 4 points in order to measure the depth diameter and the width diameter of the tumor, and the operation is troublesome to the user.

According to the ultrasound system described in Japanese Patent Application Laid-Open Publication No. 2005-193017, discrimination between those which are the target of measurement and those which are not the target of measurement cannot be made, and the depth/width ratio is an approximated value (value with the tumor as an oval). Therefore, the accuracy of measurement is not sufficient.

### BRIEF SUMMARY OF THE INVENTION

The present invention has been made in consideration of the above problems, and one of the main objects is to reduce burden of operation and to enhance accuracy of measurement in feature amount measurement of a measuring target.

According to one aspect of the present invention, there is provided an ultrasound image diagnostic apparatus which transmits and receives ultrasound with an ultrasound probe which transmits to an object transmitting ultrasound in response to a driving signal and receives reflected ultrasound to generate a receiving signal, the ultrasound image diagnostic apparatus including: a transmitter which supplies a driving signal to a transducer of the ultrasound probe; a receiver which generates acoustic ray data based on a receiving signal received from the transducer; an image generator which generates tomographic image data of the object from the generated acoustic ray data; a measuring target acknowledging unit which acknowledges a measuring target of the object from the generated tomographic image data is stably drawn for a predetermined amount of time and when it is acknowledged, advances to a measuring performing mode of the measuring target; an initial condition obtaining unit which obtains initial condition information for extracting an outline of the measuring target when advancing to the measuring performing mode; an outline extractor which uses the obtained initial condition information to extract an outline of the measuring target from the generated tomographic image data; and a measuring unit which obtains diameter information of the measuring target based on the extracted outline and calculates the feature amount of the measuring target from the diameter information.

Preferably, in the ultrasound image diagnostic apparatus, the initial condition obtaining unit receives input of the initial condition information for extracting the outline and obtains the initial condition information.

Preferably, in the ultrasound image diagnostic apparatus, the initial condition obtaining unit obtains the initial condition information for extracting the outline from the generated tomographic image data.

Preferably, in the ultrasound image diagnostic apparatus, the outline extractor uses the obtained initial condition information to extract a first outline of the measuring target from the generated tomographic image data; and based on the extracted first outline, the measuring unit obtains a first diameter and a second diameter orthogonal to the first diameter as diameter information of the measuring target, and calculates a depth/width ratio of the measuring target as a feature amount of the measuring target from the first diameter and the second diameter.

Preferably, in the ultrasound image diagnostic apparatus, the receiver generates acoustic ray data based on a receiving signal obtained by transmitting and receiving ultrasound in a position corresponding to a maximum diameter surface of the measuring target of the object and a cross section orthogonal to the maximum diameter surface; the outline extractor uses the obtained initial condition information to extract a second outline of the measuring target from the generated tomographic image data of the maximum diameter surface and to extract a third outline of the measuring target from the tomographic image data of a cross section orthogonal to the maximum diameter surface; and the measuring unit obtains a third diameter and a fourth diameter orthogonal to the third diameter as diameter information of the measuring target based on the extracted second outline corresponding to the maximum diameter surface, obtains a fifth diameter as the diameter information of the measuring target based on the extracted third outline corresponding to a cross section orthogonal to the maximum diameter surface, and calculates a measuring target diameter as a feature amount of the measuring target from the third diameter, the fourth diameter and the fifth diameter.

Preferably, in the ultrasound image diagnostic apparatus, the outline extractor uses the obtained initial condition information to extract a fourth outline of the measuring target from the generated tomographic image data; the measuring unit obtains a sixth diameter and a seventh diameter orthogonal to the sixth diameter as diameter information of the measuring target based on the extracted fourth outline and calculates volume as the feature amount of the measuring target from the sixth diameter and the seventh diameter.

Preferably, in the ultrasound image diagnostic apparatus, the receiver generates the acoustic ray data based on a receiving signal obtained by transmitting and receiving the ultrasound in a position corresponding to a maximum diameter surface of the measuring target of the object and a cross section orthogonal to the maximum diameter surface; the outline extractor uses the obtained initial condition information to extract a fifth outline of the measuring target from the generated tomographic image data of the maximum diameter surface and to extract a sixth outline of the measuring target from the tomographic image data of the cross section orthogonal to the maximum diameter surface; and the measuring unit obtains an eight diameter and a ninth diameter orthogonal to the eight diameter and a ninth diameter orthogonal to the eight diameter as the diameter information of the measuring target based on the extracted fifth outline corresponding to the maximum diameter surface, obtains a tenth diameter as the diameter information of the measuring target based on the extracted sixth outline corresponding to a cross section orthogonal to the maximum diameter surface, and calculates volume as the feature amount of the measuring target from the eight diameter, the ninth diameter, and the tenth diameter.

Preferably, in the ultrasound image diagnostic apparatus, the outline extractor extracts the outline by the graph cut method based on the initial condition information and the tomographic image data.

Preferably, in the ultrasound image diagnostic apparatus, the initial condition information is position information of a point for setting a specifying region of the graph cut method, a position information of an end point of a rectangle or a straight line, or brightness information of a foreground and a background.

Preferably, in the ultrasound image diagnostic apparatus, the outline extractor extracts the outline by a dynamic outline method based on the initial condition information and the tomographic image data.

Preferably, in the ultrasound image diagnostic apparatus, the initial condition information is position information of a point for setting the initial outline in the dynamic outline method, position information of an end point of a rectangle or a straight line, or an initial outline.

Preferably, the ultrasound image diagnostic apparatus, further includes, an operation input unit which receives input of correction information of a position of a measuring marker of the extracted outline, wherein, the measuring unit obtains diameter information of the measuring target from the measuring marker corrected by the input correction information and calculates a feature amount of the measuring target from the diameter information.

Preferably, the ultrasound image diagnostic apparatus further includes a first display controller which sets a predetermined region with the measuring marker in the initial state as the center, generates tomographic image data including the predetermined region and a moving measuring marker based on the correction information of the movement of a position of the measuring marker in the outline input from the operation input unit and displays the tomographic image data live on the display, wherein when the moving measuring marker is within the predetermined region, the moving measuring marker is displayed with the movement amount of the measuring marker made smaller for each unit of time of the operation input unit compared to when the moving measuring marker is outside the predetermined region.

Preferably, the ultrasound image diagnostic apparatus further includes a second display controller which generates tomographic image data including a moving measuring marker based on the correction information of the movement of a position of the measuring marker in the outline input from the operation input unit and displays the tomographic image data live on the display, wherein when brightness gradient information of the tomographic image data in the position of the moving measuring marker is a predetermined threshold value or more, the moving measuring marker is displayed with the movement amount of the measuring marker made smaller for each unit of time of the operation input unit compared to when the brightness gradient information is smaller than the predetermined threshold value.

Preferably, the ultrasound image diagnostic apparatus further includes a third display controller which generates tomographic image data including a moving measuring marker based on correction information of the movement of a position of the measuring marker in the outline input from the operation input unit and displays the tomographic image data live on the display, wherein display is performed moving a plurality of measuring markers together in a same direction or an enlarging/reducing direction according to input of the correction information of one measuring marker.

Preferably, the ultrasound image diagnostic apparatus further includes a fourth display controller which displays display information showing switching to the measuring performing mode on the display when the mode switches to the measuring performing mode.

Preferably, the ultrasound image diagnostic apparatus further includes an output controller which outputs to an output unit the feature amount of the calculated measuring target.

Preferably, in the ultrasound image diagnostic apparatus, the measuring target acknowledging unit calculates a difference of an entire frame or a predetermined portion in the frame of a plurality of generated tomographic image data, and switches to the measuring performing mode when the calculated difference value is a predetermined threshold value or less.

According to another aspect of the present invention, there is provided an ultrasound image diagnostic apparatus which transmits and receives ultrasound with an ultrasound probe which transmits to an object transmitting ultrasound in response to a driving signal and receives reflected ultrasound to generate a receiving signal, the ultrasound image diagnostic apparatus including: a transmitter which supplies a driving signal to a transducer of the ultrasound probe; a receiver which generates acoustic ray data based on a receiving signal from the transducer; an image generator which generates tomographic image data of the object from the generated acoustic ray data; an initial condition obtaining unit which obtains initial condition information for extracting an outline of a measuring target of the object; an outline extractor which uses the obtained initial condition information to extract an outline of the measuring target from the generated tomographic image data; and a measuring unit which obtains diameter information of the measuring target based on the extracted outline and calculates the feature amount of the measuring target from the diameter information.

According to another aspect of the present invention, there is provided an ultrasound image diagnostic apparatus which transmits and receives ultrasound with an ultrasound probe which transmits to an object transmitting ultrasound in response to a driving signal and receives reflected ultrasound to generate a receiving signal, the ultrasound image diagnostic apparatus including: a transmitter which supplies a driving signal to a transducer of the ultrasound probe; a receiver which generates acoustic ray data based on a receiving signal received from the transducer; an image generator which generates tomographic image data of the object from the generated acoustic ray data; an initial condition obtaining unit which obtains initial condition information for extracting an outline of a measuring target of the object; an outline extractor which uses the obtained initial condition information to extract an outline of the measuring target from a plurality of frames of the generated tomographic image data; a measuring unit which obtains diameter information of the measuring target based on the extracted outline of the plurality of frames; and a selecting unit which selects diameter information of one frame among the obtained diameter information of the plurality of frames, wherein the measuring unit calculates a feature amount of the measuring target from the selected diameter information.

Preferably, in the ultrasound image diagnostic apparatus, the selecting unit automatically selects diameter information with a maximum diameter among the obtained diameter information of the plurality of frames.

Preferably, in the ultrasound image diagnostic apparatus, the selecting unit receives input of selection of diameter information of one frame among the obtained diameter information of the plurality of frames.

### BRIEF DESCRIPTION OF THE DRAWINGS

The present invention will become more fully understood from the detailed description given hereinbelow and the appended drawings, and thus are not intended to define the limits of the present invention, and wherein;

FIG. 1 is an external view of a first ultrasound image diagnostic apparatus of a first embodiment of the present invention;

FIG. 2 is a block diagram showing a functional configuration of the first ultrasound image diagnostic apparatus;

FIG. 3A is a diagram showing diameter information of a tumor measured in the depth/width ratio measuring mode;

FIG. 3B is a diagram showing diameter information of a tumor measured at a maximum diameter surface in a tumor diameter measuring mode;

FIG. 3C is a diagram showing diameter information of a tumor measured in a cross section orthogonal to a maximum diameter surface in a tumor diameter measuring mode;

FIG. 4 is a flowchart showing a tumor depth/width ratio measuring processing;

FIG. 5 is a schematic diagram showing a first display image specifying a center point of the tumor;

FIG. 6 is a schematic diagram showing a second display image positioning a measuring marker;

FIG. 7A is a schematic diagram showing a third display image specifying ROI;

FIG. 7B is a schematic diagram showing a fourth display image specifying a straight line;

FIG. 8 is a flowchart showing tumor diameter measuring processing;

FIG. 9 is a schematic diagram showing a fifth display image including a display image of a maximum diameter surface of the tumor and a display image of a cross section orthogonal to the maximum diameter surface;

FIG. 10 is a block diagram showing a functional configuration of a second ultrasound image diagnostic apparatus of a second embodiment;

FIG. 11 is a schematic diagram showing a sixth display image including a B mode image including a urinary bladder;

FIG. 12 is a flowchart showing a first measuring target volume measuring processing;

FIG. 13 is a schematic diagram showing a seventh display image including a B mode image of a cross section including a urinary bladder;

FIG. 14 is a flowchart showing a second measuring target volume measuring processing;

FIG. 15 is a schematic diagram showing an eighth display image including a B mode image of a cross section orthogonal to the seventh display image shown in FIG. 13;

FIG. 16 is a schematic diagram showing a ninth display image including a B mode image including a urinary bladder;

FIG. 17A is a diagram showing a brightness profile of a reference line in a vertical direction of one cross section; and

FIG. 17B is a diagram showing a brightness profile of a reference line in a horizontal direction from a reference point of a reference line in a vertical direction as shown in FIG. 17A.

### DETAILED DESCRIPTION OF PREFERRED EMBODIMENTS

First and second embodiments and a modification of the present invention are described in detail in order with reference to the attached drawings. The present invention is not limited to the illustrated examples.

### First Embodiment

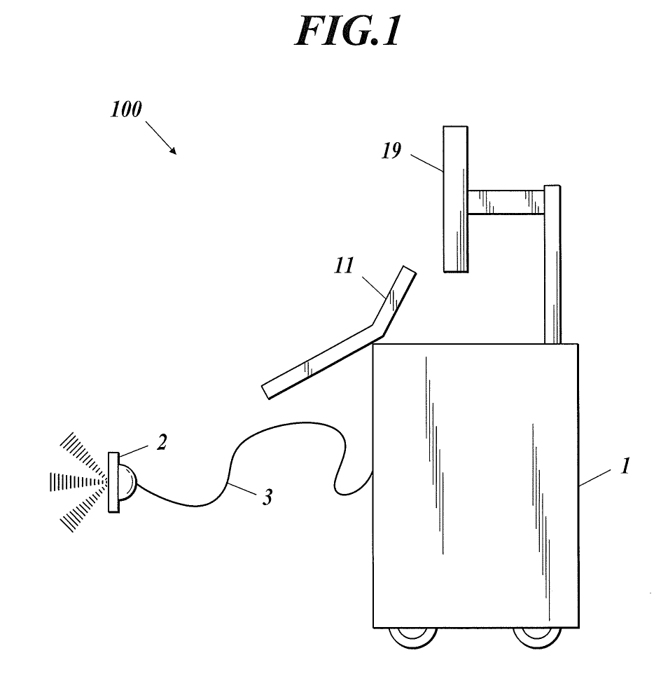

The first embodiment of the present invention is described with reference to FIG. 1 to FIG. 9. First, the entire configuration of an ultrasound image diagnostic apparatus as the ultrasound image processing apparatus of the present embodiment is described with reference to FIG. 1. FIG. 1 is an external view of the ultrasound image diagnostic apparatus 100 of the present embodiment.

As shown in FIG. 1, the ultrasound image diagnostic apparatus 100 includes an ultrasound image diagnostic apparatus main body 1 and an ultrasound probe 2. The ultrasound probe 2 transmits ultrasound (transmitting ultrasound) to a subject such as a live body (not shown) and receives ultrasound reflected from the subject (reflecting ultrasound: echo). The ultrasound image diagnostic apparatus main body 1 is connected to the ultrasound probe 2 through the cable 3. The ultrasound image diagnostic apparatus main body 1 transmits an electric driving signal to the ultrasound probe 2 so that the ultrasound probe 2 transmits the transmitting ultrasound to the object. Further, based on the receiving signal which is an electric signal generated in the ultrasound probe 2 according to the reflecting ultrasound from the object received in the ultrasound probe 2, the internal state of the object is imaged as the ultrasound image. The ultrasound image diagnostic apparatus main body 1 includes the later-described operation input unit 11 and display 19.

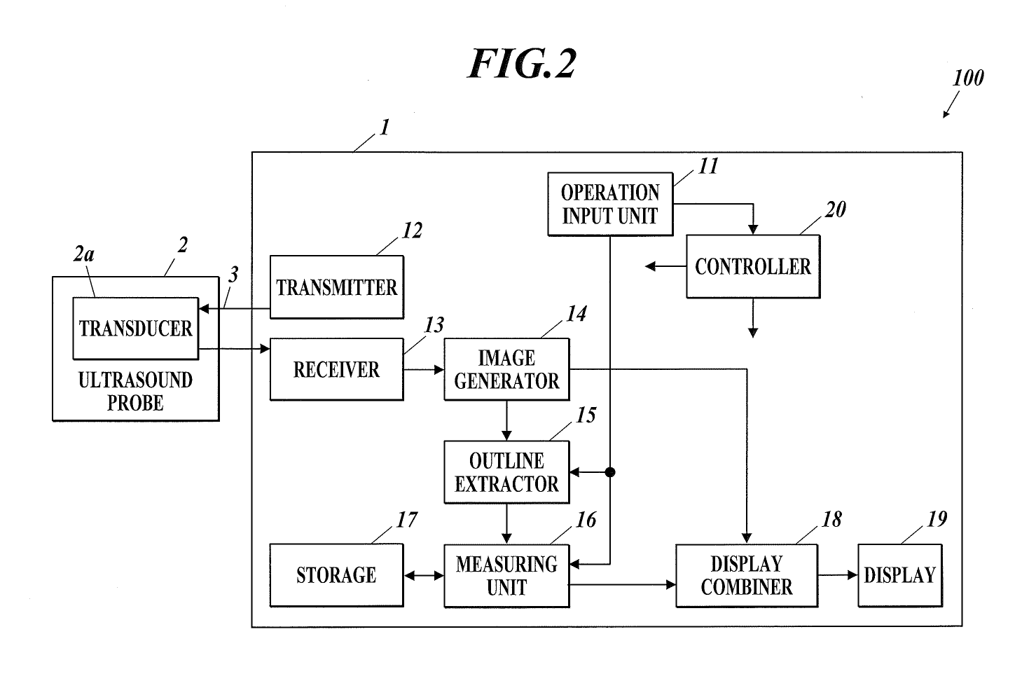

The ultrasound probe 2 includes a transducer 2a (see FIG. 2) including a piezoelectric element. For example, a plurality of transducers 2a are arranged in a one-dimensional array in an orientation direction (scanning direction). According to the present embodiment, for example, the ultrasound probe 2 including 192 transducers 2a is used. The transducers 2a can be arranged in a two-dimensional array. The number of transducers 2a can be set freely. According to the present embodiment, a linear electronic scanning probe is used as the ultrasound probe 2 to perform ultrasound scanning by a linear scanning method. Alternatively, a sector scanning method or convex scanning method can be employed. The communication between the ultrasound image diagnostic apparatus main body 1 and the ultrasound probe 2 can be performed by wireless communication such as Ultras Wide Band (UWB) instead of wired communication through the cable 3.

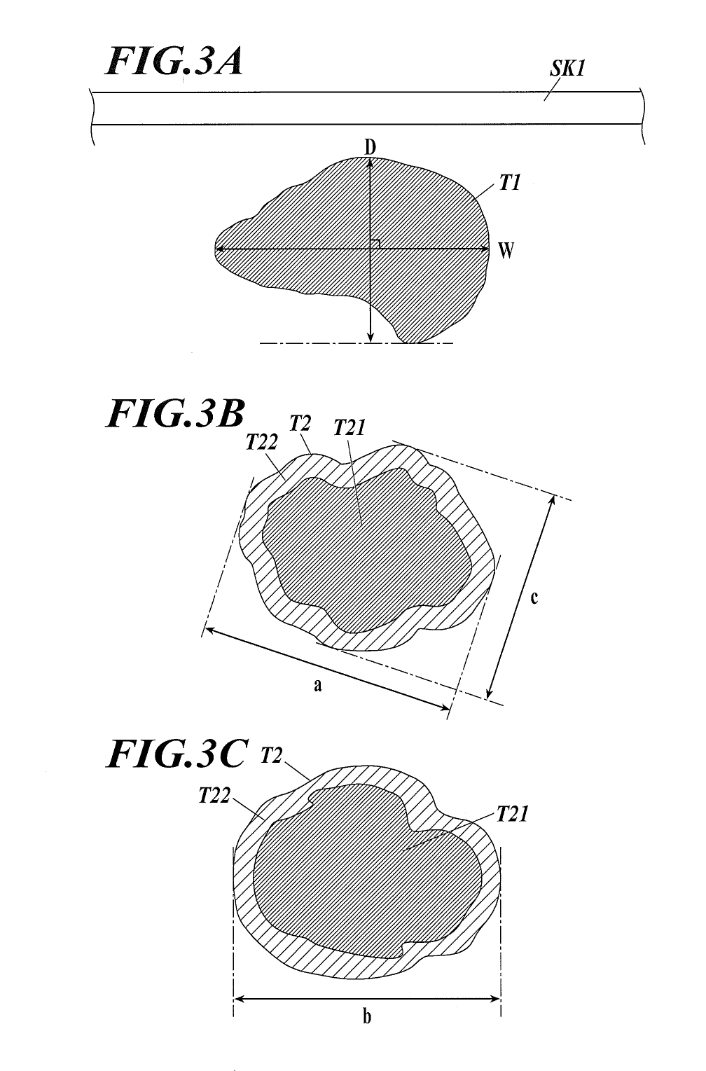

Next, the functional configuration of the ultrasound image diagnostic apparatus 100 is described with reference to FIG. 2 to FIG. 3C. FIG. 2 is a block diagram showing the functional configuration of the ultrasound image diagnostic apparatus 100. FIG. 3A is a diagram showing diameter information of the tumor as a measuring target of an object to be measured in a depth/width ratio measuring mode. FIG. 3B is a diagram showing diameter information of a tumor measured at a maximum diameter surface in the tumor diameter measuring mode. FIG. 3C is a diagram showing diameter information of the tumor to be measured at a cross section orthogonal to the maximum diameter surface in the tumor diameter measuring mode.

As shown in FIG. 2, for example, the ultrasound image diagnostic apparatus main body 1 includes, an operation inputting unit 11, a transmitter 12, a receiver 13, an image generator 14, an outline extractor 15, a measuring unit 16, a storage 17, a display combiner 18 as a first and second display controlling unit and output controlling unit, a display 19 as an output unit, and a controller 20.

The operation input unit 11 includes, for example, various switches, buttons, a track ball, a mouse, a keyboard, a touch panel provided as one with the display screen of the display 19, etc. to input a command to instruct start of diagnosis, data such as personal information of the object, and various parameters to display the ultrasound image on the display 19. The operation input unit 11 outputs the operation signal to the controller 20. Specifically, the operation input unit 11 receives input of specification of depth/width ratio measuring mode or tumor diameter measuring mode, outline extracting initial condition information, and correction information of position of measuring marker in diameter information of the tumor.

Here, the measurement mode is described with reference to FIG. 3A and FIG. 3B. The depth/width ratio measuring mode is a measuring mode mainly used in examination. As shown in FIG. 3A, the maximum width diameter W parallel to a skin surface SK1 and a maximum depth diameter D orthogonal to the maximum width diameter W are measured for a tumor T1 not including halo, and a depth/width ratio as a feature amount of the tumor T1 is calculated by the following formula (1) and displayed.

depth/width ratio=D/W (1)

Halo in the field of mammary gland is a boundary high echo portion around a low echo portion of the tumor. The boundary for measurement of the tumor not including the halo in the depth/width ratio measuring mode is a boundary in which difference in brightness from the surrounding area (area which is not the tumor) is larger than when the halo is included as described later. Therefore, the tumor T1 includes only the low echo portion as the tumor main body portion. End points of the width diameter W and the depth diameter D are set on an outline of the low echo portion of the tumor or on an extension line of the outline (for example, extension line of the outline of the depth diameter D shown in FIG. 3A).

The tumor diameter measuring mode is a measuring mode used when there is effect over time in preoperative chemotherapy. As shown in FIG. 3B, a maximum width diameter a and a maximum depth diameter c of a tumor T2 in a maximum diameter surface which is a cross section in which the diameter of the tumor T2 is largest is measured for the tumor T2 including the low echo portion T21 as the tumor main body portion and the halo T22. As shown in FIG. 3C, the maximum width diameter b of the tumor T2 in the cross section orthogonal to the maximum diameter surface of the tumor T2 is measured, and the tumor diameter as the feature amount of the tumor T2 is calculated by the following formula (2) and displayed.

tumor diameter=a*b*c (2)

the unit of a, b, c is [mm] or [cm].

The boundary of the measurement of the tumor including the halo in the tumor diameter measuring mode is the boundary which can be confirmed by sight but which has a brightness difference from the surroundings which is smaller than the brightness difference not including the halo. In the diameter measuring mode, measurement of two cross sections is necessary. The tumor diameter of the tumor calculated in the tumor diameter measuring mode is often used as a value compared to the past measurement value (calculating value). The end points of the width diameters a, b and depth diameter c are set on the outline of the halo of the tumor or on the extension line of the outline.

Returning to FIG. 2, under the control of the controller 20, the transmitter 12 is a circuit which provides an electric driving signal through the cable 3 to the ultrasound probe 2 so that the ultrasound probe 2 generates the transmitting ultrasound. For example, the transmitter 12 includes a clock generating circuit, a delaying circuit, and a pulse generating circuit. The clock generating circuit is a circuit which generates a clock signal to determine transmission timing of the driving signal and the transmission frequency. The delay circuit is a circuit which sets the delay time for each individual path corresponding to each transducer 2a and delays the transmission of the driving signal in the set amount of delay time to focus the transmission beam composed of the transmitting ultrasound (transmission beam forming). The pulse generating circuit is a circuit to generate the pulse signal as the driving signal with the set voltage and time interval. Under the control of the controller 20, the transmitter 12 as described above sequentially switches the plurality of transducers 2a to which the driving signal is provided, shifting a predetermined number each time the ultrasound is transmitted and received, and provides the driving signal to the plurality of transducers 2a with the selected output to be scanned.

The receiver 13 is a circuit which receives the electric receiving signal through the cable 3 from the ultrasound probe 2 according to the control of the controller 20. For example, the receiver 13 includes an amplifier, an A/D conversion circuit, and a phasing/adding circuit. The amplifier is a circuit to amplify the receiving signal at a preset amplifying rate for each individual path corresponding to each transducer 2a. The A/D conversion circuit is a circuit for analog-digital conversion (A/D conversion) on the amplified receiving signal. The phasing/adding circuit is a circuit which provides a delay time for each individual path corresponding to each transducer 2a on the A/D converted receiving signal to adjust the time phase, and adds the above to generate acoustic ray data. That is, the phasing/adding circuit performs reception beam forming on the receiving signal for each transducer 2a and generates the acoustic ray data.

According to the control of the controller 20, the image generator 14 performs on the acoustic ray data from the receiver 13 envelope detecting processing and logarithmic compression and performs adjustment of dynamic range and gain to convert the brightness. With this, the image generator 14 generates B (Brightness) mode image data as tomographic image data. That is, the B mode image data represents the strength of the receiving signal with brightness.

The image generator 14 includes an image memory (not shown) composed of a semiconductor memory such as a DRAM (Dynamic Random Access Memory). The image generator 14 stores the generated B mode image data in the image memory in a unit of frames.

The image generator 14 suitably performs image processing such as image filter processing and time smoothing processing on the ultrasound image data read out from the image memory and scans and converts the data to a display image pattern to be displayed on the display 19.

According to control of the controller 20, the outline extractor 15 uses the predetermined image processing method to extract the outline of the tumor of the object from the B mode image data generated in the image generator 14 and outputs the outline data. According to the present embodiment, a dynamic outlining method such as a Snakes method is used as an example of the image processing method of the outline extractor 15.

The Snakes method is a method of extracting the outline by using an energy function represented as a linear sum of the internal modifying energy and external potential energy on the curve of an image plane, and modifying the shape so that the energy function is minimum.

That is, a closed curve (boundary line) is determined to minimize the energy function E(v) provided by the following formula (3).

E(v)=S(v)+P(v) (3)

S(v) is internal modifying energy. P(v) is external potential energy {v(s)=[x(s), y(s)]}. s is arc length parameter of the closed curve.

Specifically, an initial outline is set, local energy Esnakes represented by the following formula (4) is calculated for the plurality of pixels near one outline point (peak) on the initial outline and the smallest point is set as the new outline point.

Esnakes=αEint+βEimage (4)

Eint is internal modifying energy of the outline. Eimage is image energy showing goodness of fit between the outline and the image. α and β are parameters for weighting each energy.

The internal modifying energy Eint is represented by the following formula (5).

Eint=(w1|vs|2+w2|vss|2)/2 (5)

w1, w2 are constants showing weight. vs shows first-order differentiation of v. vss shows second order differentiation of v. v is a parameter representation of the outline.

The image energy Eimage is represented by the following formula (6).

Eimage=−(Gσ*∇2I)2 (6)

Gσ is a Gussian filter. ∇2 is a Laplacian filter. I is the brightness value.

Processing to calculate the local energy Esnakes in the plurality of pixels near one outline point and to set the new outline point in the plurality of pixels near the one outline point is performed for all outline points on the outline and the outline formed by the new outline point is set. The processing to set the new outline is repeated until a predetermined condition is satisfied. Such predetermined condition includes, for example, the entire movement amount of the outline point on the outline is a predetermined threshold value or less, or the number of times that the setting of the outline is repeated exceeds a predetermined threshold value.

As described above, the outline extractor 15 extracts the outline of the tumor in the B mode image by the Snakes method. The tumor tends to be a complicated shape, and therefore, preferably, the shape is not fixed and processing is performed with the parameter β being weighted larger than the parameter α.

The image processing method by the outline extractor 15 is not limited to the Snakes method, and other image processing methods such as a graph cut method can be used. The graph cut method extracts the boundary (outline) so that the energy function E(X) represented by the following formula (7) becomes smallest.

$$E ( X ) = {\sum }_{i ∈ v} E 1 ( xi ) + {\sum }_{( i , j ) ∈ ɛ} E 2 ( xi , xj ) \qquad ( 7 )$$

X, xi, and xj are labels. E1 is a feature amount similar to the Eimage of the Snakes method, and shows the adaptability between the pixel and the object (tumor) (for example, similarity with the object regarding the color (brightness)). E2 is a feature amount similar to Eint of the Snakes method and is a feature amount which functions as the role to fix the shape (for example, shows the relation between the adjacent boundary points). v is a set of places (sites). ε is a set of pairs of adjacent places.

Specifically, the label is a label showing whether it is a tumor or not. The site is the pixel. The adjacent relation is the adjacent relation of the pixels. The first term of the right side of the formula (7) is a data term and is a term showing whether it may be a tumor or not from the color of the pixel (brightness value). The second term of the right side of the formula (7) is a smoothing term and is a term which smoothes the label between the adjacent pixels.

Then, based on the energy function E(X) of the formula (7), the outline extractor 15 makes a graph. The graph includes 2 terminals, a source and a sink showing the label of whether it is a tumor or not, a plurality of nodes corresponding to the plurality of pixels, and links between the terminal and each node. A cost (energy) of cutting is set in each link. Then, the outline extractor 15 cuts the graph between the 2 terminals so that the cost becomes smallest (energy function E(X) is minimum) and the cut surface is to be the outline of the tumor. With the graph cut method, the outline of the object other than the tumor may be extracted. In such case, the largest among the plurality of outlines is extracted as the outline of the tumor.

Under the control of the controller 20, the measuring unit 16 calculates position information of a measuring marker for measuring diameter information of the tumor, diameter information, and a feature amount based on the diameter information from the outline data input from the outline extractor 15, outputs the position information of the measuring marker, the diameter information, and the feature amount to the display combiner 18, and stores the diameter information and the feature amount in the storage 17.

In the depth/width ratio measuring mode, the position information of the measuring marker is the intersection point where the width diameter and the depth diameter intersects with the outline of the tumor based on the outline data or the extension line of the tumor. The diameter information is the width diameter W and the depth diameter D. The feature amount is the depth/width ratio of the tumor.

In the tumor diameter measuring mode, the position information of the measuring marker is the intersection point where the width diameter a and the depth diameter c of the maximum diameter surface and the width diameter b of the cross section orthogonal to the maximum diameter surface intersect with the outline of the tumor based on the outline data of each cross section or the extension line of the outline. The diameter information includes the width diameters a and b, and the depth diameter c. The feature amount is the tumor diameter.

The storage 17 is a storage unit which is able to write and read out information in devices such as a flash memory, a HDD (Hard Disk Drive), a SSD (Solid State Drive), etc.

Under the control of the controller 20, the display combiner 18 generates the display image data by combining as is or suitably processing the B-mode image data input from the image generator 14 with the measuring marker according to the position information of the measuring marker, the diameter information, and the feature amount output from the measuring unit 16. The display combiner 18 outputs the image signal of the display image data to the display 19.

Display apparatuses such as a LCD (Liquid Crystal Display), a CRT (Cathode-Ray Tube) display, an organic EL (Electronic Luminescence) display, an inorganic EL display, a plasma display or the like can be applied as the display 19. According to the image signal of the display image data input from the display combiner 18, the display 19 displays the display image on the display screen.

For example, the controller 20 includes a CPU (Central Processing Unit), a ROM (Read Only Memory), and a RAM (Random Access Memory). The controller 20 reads out various processing programs such as a system program stored in the ROM, develops the program in the RAM, and centrally controls the operation of each unit of the ultrasound image diagnostic apparatus main body 1 according to the developed program. The ROM includes a nonvolatile memory such as a semiconductor, etc., and stores the system program corresponding to the ultrasound image diagnostic apparatus 100 and various processing programs which can be executed on the system program, and various data such as a gamma table. Such programs are stored in a form of a computer readable program code, and the CPU sequentially performs operation according to the program code. The RAM forms a work area which temporarily stores various programs executed by the CPU and data regarding the program. Specifically, the ROM of the controller 20 stores the tumor depth/width ratio measuring program and the tumor diameter measuring program. The controller 20 controls each unit of the ultrasound image diagnostic apparatus main body 1, but lines showing control are omitted in FIG. 2.

Part of or all functions of each functional block provided in the ultrasound image diagnostic apparatus 100 such as the transmitter 12, the receiver 13, the image generator 14, the outline extractor 15, the measuring unit 16, the display combiner 18, and the controller 20 can be realized with a hardware circuit such as an integrated circuit. An integrated circuit is, for example, an LSI (Large Scale Integration), and the LSI may be called an IC, a system LSI, a super LSI, or an ultra LSI depending on the degree of integration. The method of integrating in a circuit is not limited to the LSI and can be realized by a dedicated circuit or general purpose processor. A FPGA (Field Programmable Gate Array) or a reconfigurable processor in which connection and setting of a circuit cell in the LSI can be reconfigured can also be used. Alternatively, part of or all functions of each functional block can be executed by software. In this case, the software is stored in at least one storage medium such as a ROM, optical disk, or hard disk, and the software is executed by an arithmetic processor. The above is similarly applied to each unit in an ultrasound image diagnostic apparatus 100A of the second embodiment. Part of or all functions of the measuring object recognizing unit 21 of the ultrasound image diagnostic apparatus 100A of the second embodiment can also be realized by the hardware circuit such as the integrated circuit.

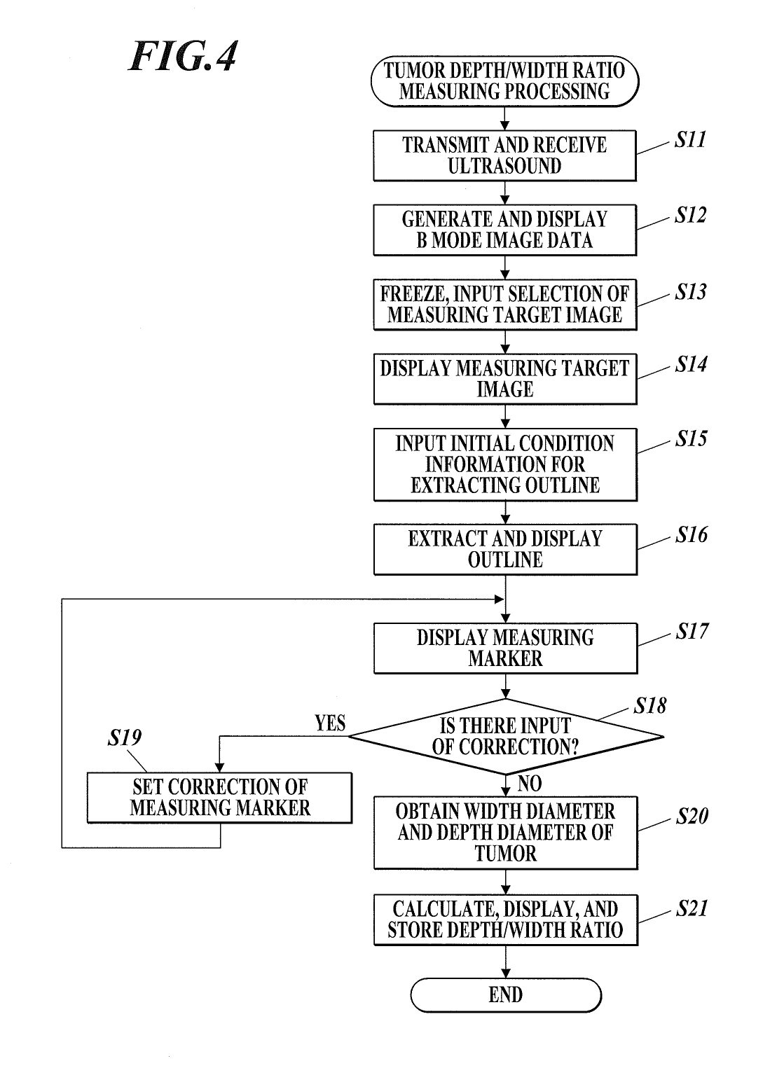

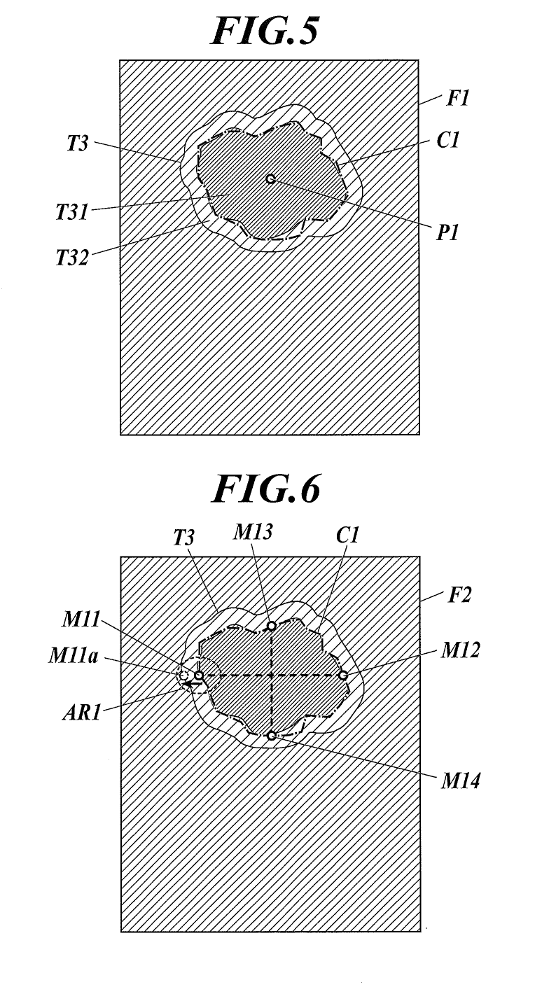

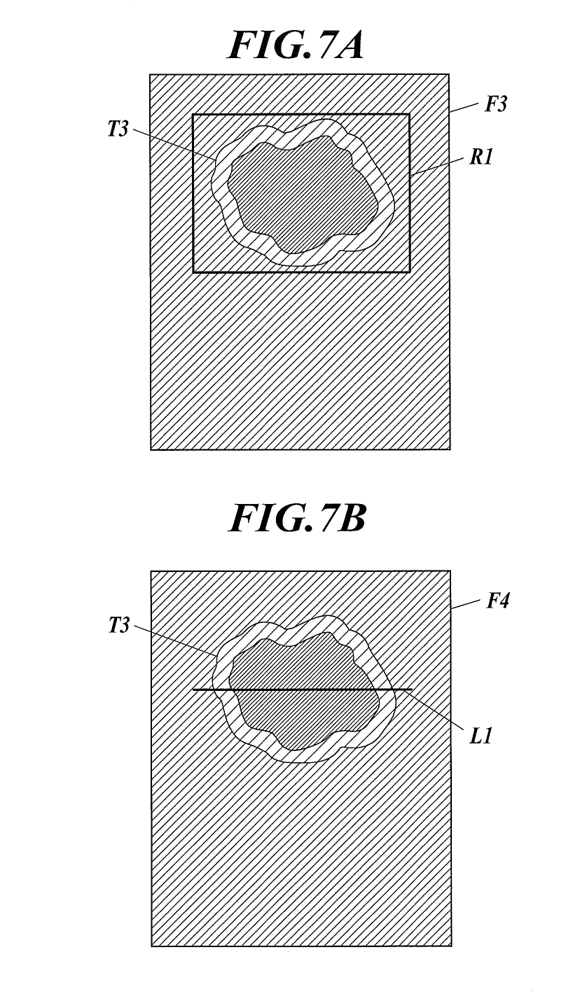

Next, the operation of the ultrasound image diagnostic apparatus 100 is described with reference to FIG. 4 to FIG. 9. Specifically, the tumor depth/width ratio measuring processing and the tumor diameter measuring processing performed in the controller 20 are described. First, with reference to FIG. 4 to FIG. 7, the tumor depth/width ratio measuring processing is described. FIG. 4 is a flowchart showing the tumor depth/width ratio measuring processing. FIG. 5 is a schematic diagram showing a display image F1 specifying, for example, the center point P1 of the tumor. FIG. 6 is a schematic diagram showing a display image F2 provided with the measuring markers M11, M12, M13, and M14. FIG. 7A is a schematic diagram showing a display image F3 specifying a ROI (Region of Interest) RE FIG. 7B is a schematic diagram showing a display image F4 specifying a straight line L1.

The tumor depth/width measuring processing is processing which obtains the depth diameter and the width diameter of the tumor from the B mode image data obtained by receiving ultrasound and calculates the depth/width ratio. For example, when the user (engineer, physician, etc.) as the tester of the object inputs and specifies the tumor depth/width ratio measuring mode through the operation/input unit 11, this triggers the controller 20 to execute the tumor depth/width measuring processing by controlling each unit according to the tumor depth/width ratio measuring program stored in the ROM.

As preparation to measure the depth/width ratio of the tumor, the user operates the ultrasound probe 2 and places the ultrasound probe 2 against the object which has the tumor. Then, as shown in FIG. 4, the transmitter 12 and the receiver 13 transmits and receives the ultrasound for the B mode image through the ultrasound probe 2 in the unit of frames (step S11).

Then, the image generator 14 generates the B mode image data corresponding to the ultrasound transmission and reception in step S11, and outputs the data to the outline extractor 15 and the display combiner 18. The display combiner 18 displays a live B mode image on the display 19 based on the input B mode image data (step S12).

The user confirms the image including the maximum width diameter of the measuring target in the displayed B mode image. Then, the operation/input unit 11 receives input from the user to freeze the live B mode image. The display combiner 18 displays on the display 19 the plurality of frames (cine-frame) of B mode image data before the freezing operation in response to freezing operation. The operation/input unit 11 receives from the user the input of selection of the measuring target image (B mode image of the frame with the measuring target) among the B mode image of the plurality of frames (step S13). Then, the display combiner 18 displays on the display 19 the measuring target image based on the B mode image data of the measuring target image input in step S13 (step S14).

Then, the operation/input unit 11 receives the initial condition information to extract the outline from the user (step S15). The initial condition information to extract the outline is position information of one point specified by the user and this is used as the position information of the center point of the initial outline in the Snakes method. This point is to be the point for initial outline setting. In order to enhance the accuracy of the extracting the outline, preferably, the user specifies a point near the center of the tumor. The parameters α, β of the above formula (4) of the Snakes method is stored in the storage 17 in advance.

Then, the outline extractor 15 uses the initial condition information input in step S13 and the parameters α, β stored in the storage 17 and extracts the outline of the tumor not including the halo from the B mode image data of the measuring target image selected in step S13 to generate the outline data. The outline data is output to the display combiner 18 through the measuring unit 16. The display combiner 18 generates combined image data including the outline on the B mode image based on the input B mode image data, the position information of the point of the initial outline setting, and the outline data. The combined image based on the combined image data is displayed on the display 19 (step S16). In step S16, the outline extractor 15 sets as the initial outline the circle or the oval with the predetermined diameter including the point for initial outline setting in the initial condition information as the center point, and extracts the outline from the B mode image data based on the initial outline by the Snakes method. According to such configuration, since the initial outline is set with the point input as the initial condition information as the center, the burden of operation on the user is reduced.

Typically, since the tumor has a low brightness compared to the surrounding tissue, when the outline of the tumor not including the halo is extracted, the outline candidate is searched outward from the center of the initial outline and the points which change from the low brightness to the high brightness is extracted to set the image energy Eimage.

For example, in step S16, the display image F1 shown in FIG. 5 is displayed. The display image F1 is a B mode image scanning the tumor T3, and is combined with the center point P1 and the outline C1. The tumor T3 includes a low echo portion T31 as the tumor main body portion and a halo T32 as a high echo portion surrounding the low echo portion T31. The outline C1 is generated to match the outline of the low echo portion T31.

Then, the position information of the 4 measuring markers as the end points of the width diameter and the depth diameter for the depth/width ratio of the tumor generated by the measuring unit 16 using the outline data extracted in step S13 or the position information of the measuring marker modified in the later described step S19 is used so that the display combiner 18 generates the combined image data based on the input B mode image data, the outline data and the generated position information of the measuring marker, and the combined image based on the combined image data including the outline on the B mode image and the measuring marker is displayed on the display 19 (step S17).

In step S17, for example, the display image F2 shown in FIG. 6 is displayed. The display image F2 is a B mode image scanning the tumor T3, and the outline C1 and the measuring markers M11, M12, M13, and M14 are combined. In the initial state, the measuring markers M11 and M12 are positioned in the intersecting point of the outline C1 and the straight line parallel with the upper side of the display image F2 as the skin surface of the object and including the maximum width diameter of the tumor T3. The measuring markers M13 and M14 are positioned in the intersecting point of the outline C1 and the straight line which is orthogonal to the straight line connecting the measuring markers M11 and M12 and which includes the maximum depth diameter of the tumor T3. Since the measuring markers M11, M12, M13, and M14 include the maximum width diameter and the depth diameter of the tumor T3, the measuring markers can be positioned in the intersecting point of each straight line and the extension line of the outline C1.

Then, the controller 20 determines whether the correction information of the position of the measuring marker is input by the user through the operation/input unit 11 (step S18). When the correction information is input (step S18; YES), the measuring unit 16 corrects the position information of the measuring marker generated in step S17 based on the correction information input in step S18 (step S19), and the process advances to step S17.

For example, the input of the correction information of the position of the measuring marker is performed by selecting and moving the measuring marker using the track ball, cursor button, etc. of the operation/input unit 11, and determining the correction. For example, when the measuring marker M11 is moved and the measuring marker after correction is to be the measuring marker M11a as shown in FIG. 6, the movement amount of the measuring marker M11 being moved for each unit of time of the trackball, etc. is made small in a region AR1 with a circle in a predetermined distance from the measuring marker M11 as the correction target so that fine adjustment for correction can be made. Outside the region AR1, the movement amount of the measuring marker M11 being moved for each unit of time of the trackball, etc. is made large to shorten time for correction. Specifically, the region AR1 data showing the shape and size of the region AR1 is stored in advance in the storage 17. Based on the region AR1 data stored in the storage 17, the display combiner 18 sets the region AR1 with the initial (before movement) measuring marker M11 as the center. Based on the correction information of the moved position of the measurement marker M11 input from the operation/input unit 11, the display combiner 18 generates the combined image data of the display image F2 including the region AR1, the measuring marker M11 being moved and the outline C1. The combined image data is displayed live on the display 19. When the measuring marker M11 being moved is in the region AR1, the movement amount of the track ball, etc. of the operation/input unit 11 for each unit of time is made small compared to being moved outside the region AR1, and the measuring marker M11 being moved is displayed. The shape of the region AR1 is not limited to a circle, and can be other shapes such as a rectangle. The display of the region AR1 is not required. The correction of the measuring markers M12, M13, and M14 using the predetermined region is similar to the correction of the measuring marker M11 using the region AR1.

When the brightness gradient of the B mode image near the correction target measuring marker M11 being moved is a predetermined threshold value or more, the movement amount (movement speed) of the measuring marker M11 being moved for each unit of time of the track ball, etc. can be made small. When the brightness gradient is smaller than a predetermined threshold value or there is no substantial change, the movement amount of the measuring marker M11 being moved for each unit of time of the track ball, etc. can be made large to shorten the time for correction. Specifically, a predetermined threshold value of the brightness gradient information is stored in the storage 17. Based on the correction information of the movement of the position of the measuring marker M11 input from the operation input unit 11, the display combiner 18 generates the combined image data of the display image F2 including the measuring marker M11 being moved and the outline C1 and displays the live image on the display 19. When the brightness gradient information of the B mode image data in the position of the measuring marker M11 being moved is equal to or more than a predetermined threshold value of the brightness gradient information stored in the storage 17, the movement amount of the trackball, etc., of the operation/input unit 11 for each unit of time is made smaller than when the brightness gradient information is smaller than the predetermined threshold value, and the measuring marker M11 being moved is displayed. The correction of the measuring markers M12, M13, and M14 using the brightness gradient information is similar to the correction of the measuring marker M11 using the brightness gradient information. After the position information of the measuring marker is determined, the measuring unit 16 can store in the storage 17 the brightness gradient information of the measuring marker in the movement destination, and when the measuring marker is positioned the next time, the measuring marker can be automatically moved and positioned in the position corresponding to the previously stored brightness gradient information in the B mode image.

The brightness gradient information of the measuring marker in the movement destination after correction stored in the storage 17 can be set so that in the next extraction of the outline, when the stored brightness gradient information is close, the evaluation of the image energy E image of the formula (4) increases (value decreases).

When the correction information is not input (step S18; NO), the measuring unit 16 obtains the width diameter and the depth diameter of the tumor from the present position information of the measuring markers M11, M12, M13, and M14 (step S20). Then, the measuring unit 16 uses the obtained width diameter W and depth diameter D to calculate the depth/width ratio of the tumor with the formula (1), and stores the width diameter W and the depth diameter D of the tumor and the calculated depth/width ratio of the tumor in the storage 17. The display combiner 18 displays on the display 19 the obtained width diameter W and the depth diameter D of the tumor and the calculated depth/width ratio of the tumor (step S21). With this, the tumor depth/width ratio measuring processing ends.

The initial condition information for extracting the outline input in step S15 is not limited to the position information of the center point of the initial outline. For example, as shown in FIG. 7A, the setting information of the rectangle R1 of the ROI can be input as the initial condition information in the display image F3 including the tumor T3. The rectangle R1 as the ROI is input so as to surround the tumor T3. The setting information of the rectangle R1 includes, for example, position information of the upper left point and the bottom right point of the rectangle R1, position information of two end points of the straight line connecting the center point of the rectangle R1 and one of the vertexes, position information of the center point of the rectangle R1 when the size of the rectangle R1 is set in advance, or the like. The initial outline is set by forming a circle or an oval inscribed in the rectangle R1 or by performing binarization processing on the B mode image in the rectangle R1 as the ROI and setting the boundary of the low echo region as the initial outline. Then, outline searching processing is performed in the pixel in the ROI instead of in the entire image. According to such configuration, the range of searching for the outline becomes small, and the performance of extracting the outline (accuracy and extracting speed) is expected to be enhanced.

In step S15, for example, as shown in FIG. 7B, the setting information of the straight line L1 can be input as the initial condition information in the display image F4 including the tumor T3. The straight line L1 is input so as to be parallel to the skin surface and to penetrate (cross) the tumor T3. For example, the setting information of the straight line L1 is position information of two end points of the straight line L1. The initial outline is the circle or the oval with the two end points of the straight line L1 as the diameter. Here, the intersecting point of the straight line L1 and the low echo region is extracted, and the circle or the oval passing these two points are to be the initial outline. With this, it is possible to set the initial outline which passes a point near the outline of the tumor, and the performance of extracting the outline is enhanced.

In step S16, the outline extractor 15 may calculate the outline by a graph cut method. According to such configuration, setting information of the specified region such as a rectangle, a circle, an oval, etc. is input as the initial condition information for extracting the outline in step S15. For example, the outline extractor 15 sets the average value of the brightness value of the B mode image in the specified region based on the input setting information, the brightness value with the largest number of pixels, etc. as the brightness value of the region (background region) other than the tumor. The brightness value of the center of gravity, etc. of the specified region is set in the formula (7) as the brightness value of the region of the tumor, and the set formula (7) is used to extract the outline of the graph cut method. The setting information of the specified region includes, for example, the position information of two end points of the straight line as the diameter or the radius of a circle when the specified region is a circle, or the position information such as the center point of the specified region when the shape and size of the specified region is set in advance. Although the number of times of operation increases, the operation input unit 11 may receive input from the user specifying two points, the point showing the brightness value of the region of the tumor in the B mode image and the point showing the brightness value of the background region as the initial condition information for extracting the outline, and the outline extractor 15 may extract the outline of the tumor by the graph cut method using the brightness values of the two input points.

The operation input unit 11 may receive the input from the user specifying the shape surrounding the tumor on the touch panel as the initial outline of the initial condition information according to the dynamic outline method or as the specified region according to the graph cut method.

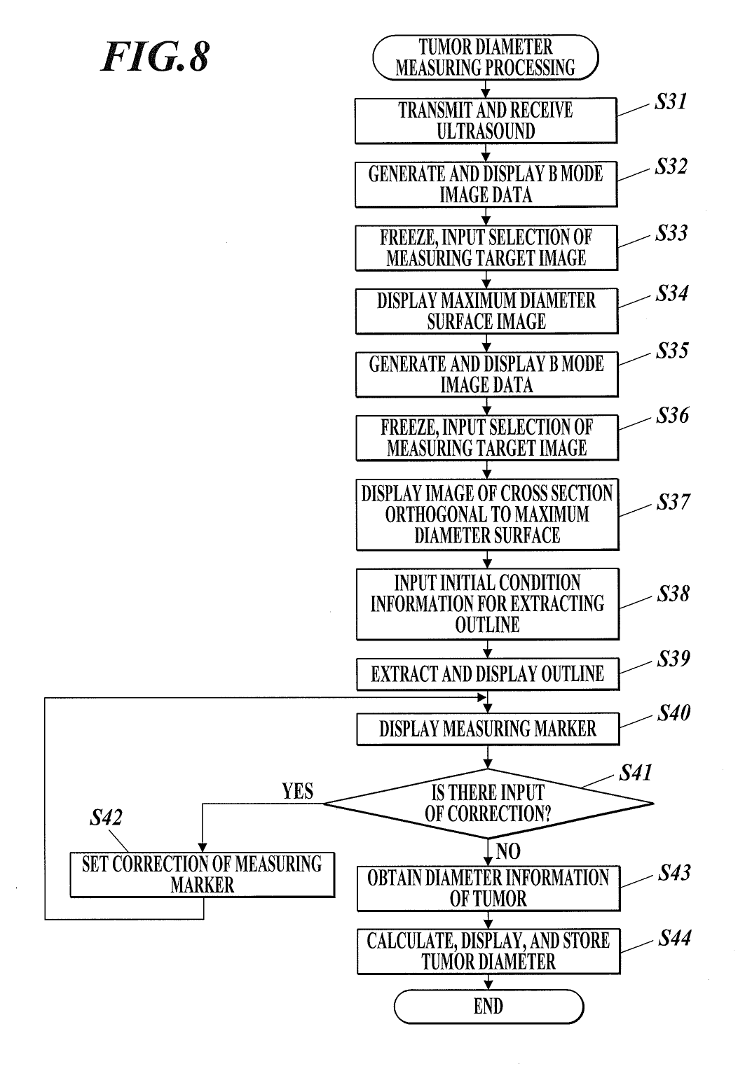

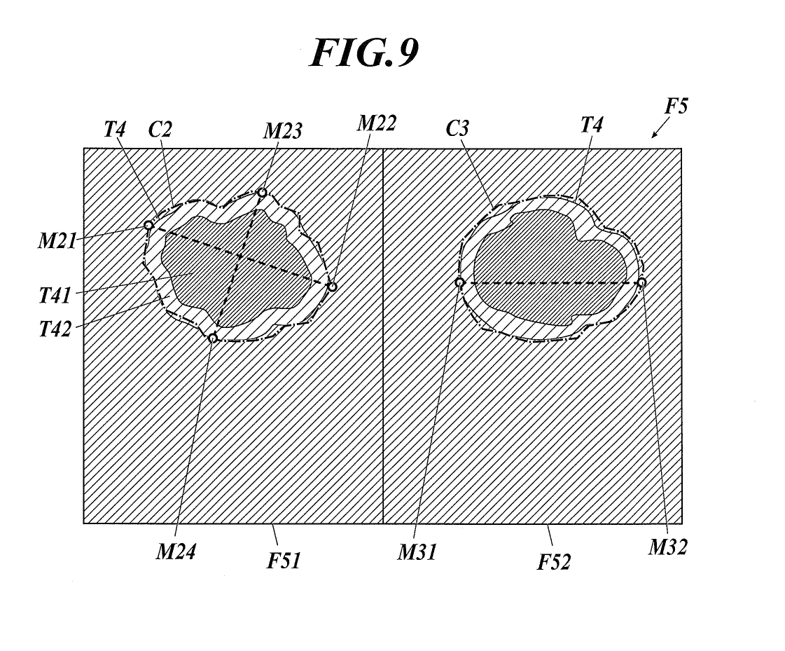

Next, the tumor diameter measuring processing is described with reference to FIG. 8 and FIG. 9. FIG. 8 is a flowchart showing the tumor diameter measuring processing. FIG. 9 is a schematic diagram showing a display image F5 including a display image F51 showing a maximum diameter surface of the tumor T4, and a display image F52 showing a cross section orthogonal to the maximum diameter surface.

According to the tumor diameter measuring processing, the width diameter and the depth diameter of the tumor is measured from the B mode image data obtained by transmitting and receiving the ultrasound to calculate the tumor diameter. For example, the user inputting and specifying the tumor diameter measuring mode through the operation input unit 11 acts as a trigger, and the controller 20 controls each section to perform the tumor diameter measuring processing according to the tumor diameter measuring program stored in the ROM.

First, in the ultrasound image diagnostic apparatus 100, similar to steps S11, S12 of the tumor depth/width ratio measuring processing shown in FIG. 4, the transmitter 12, the receiver 13, the image generator 14, and the display combiner 18 transmit and receive ultrasound, generate B mode image, and display the image (steps S31, S32). The user operates the ultrasound probe 2, places the ultrasound probe 2 against the subject including the tumor, confirms by sight the displayed B mode image while rotating the ultrasound probe 2 on the surface of the skin, and searches for the maximum diameter surface in which the diameter of the tumor becomes largest on the B mode image.

Then, similar to steps S13, S14 shown in FIG. 4, the operation input unit 11 and the display combiner 18 receive input of the freezing operation from the user at the maximum diameter surface, a plurality of frames (cine-frame) of the B mode image data is displayed, the input to select the maximum diameter surface image is received as the first measuring target image, and the selected maximum diameter surface image is displayed on the display 19 (steps S33, S34).

Next, the user releases the freezing operation. When the operation input unit 11 receives input to release the freezing operation from the user, the step S35 similar to the step S32 is performed. The user confirms the B mode image by sight again and rotates the ultrasound probe 2 on the surface of the skin so as to match with the cross section orthogonal to the maximum diameter surface. Then, similar to step S33, the operation input unit 11 and the display combiner 18 receive input of freezing operation from the user at the cross section orthogonal to the maximum diameter surface, a plurality of frames of the B mode image data are displayed, the input to select the orthogonal cross sectional image with the maximum diameter surface is received as the second measuring target image, and the selected maximum diameter surface image is displayed on the display 19 (steps S36, S37). Here, when the second measuring target image is extracted while displaying the first measuring target image on the screen, the second measuring target image can be searched easily.

Steps S38, S40 to S42 are similar to steps S15, S17 to S19 shown in FIG. 4. The input of the initial condition information for extracting the outline, the display of the measuring marker and the input of the correction information are performed for both the B mode image with the maximum diameter surface and the B mode image of the cross section orthogonal to the maximum diameter surface.

In order to extract the outline of the tumor including the halo, in step S39, first, the outline extractor 15 performs the same processing as step S16 of FIG. 4 on the B mode image data between the maximum diameter surface image and the cross section image orthogonal to the maximum diameter surface, and the outline candidate point of the tumor not including the halo is extracted. Then, the outline extractor 15 analyzes the change in brightness of the straight line connecting the center of the initial outline and the outline candidate point, and extracts the boundary point in which the brightness changes from high brightness to low brightness outside the outline candidate point of the tumor not including the halo. The outline extractor 15 performs the above on each outline candidate point of the tumor not including the halo and sets the outline of the tumor including the halo. Here, if the searching is performed by interpolating between the outline candidate points of the tumor not including the halo, although the calculating increases, the accuracy of extracting the outline is enhanced. For example, when the boundary point in which the brightness changes from high brightness to low brightness is extracted, the predetermined threshold value of the change in brightness in the boundary of the outline is stored in advance in the storage 17, and the outline extractor 15 sets the point in which the change in brightness outside the outline candidate point of the tumor not including halo on the straight line connecting the center of the initial outline and the outline candidate point is equal to or more than the predetermined threshold value stored in the storage 17 as the boundary point.

In step S40, for example, as shown in FIG. 9, the display image F5 including the tumor T4 is displayed. The display image F5 includes the display image F51 corresponding to the maximum diameter surface and the display image F52 corresponding to the cross section orthogonal to the maximum diameter surface. The display image 51 is a B mode image scanning the tumor T4, and the outline C2 is combined with the measuring markers M21, M22, M23, and M24. The tumor T4 includes a low echo portion T41 as the tumor main body portion and a halo T42 surrounding the low echo portion T41.

In the initial state, the measuring markers M21 and M22 are positioned in the intersecting point of the straight line with the maximum width diameter of the tumor T4 of the object and the outline C2. The measuring markers M23 and M24 are positioned in the intersecting point of the straight line which is orthogonal to the straight line connecting the measuring markers M21 and M22 and which includes the maximum depth diameter of the tumor and the outline C2. Since the measuring markers M21, M22, M23, and M24 include the maximum width diameter and depth diameter of the tumor T4, the measuring markers can be positioned in the intersecting point of each straight line and extension line of the outline C. The outline C2 is generated to match with the outline of the halo T42.

The display image F52 is a B mode image scanning the tumor T4, and the outline C3 is combined with the measuring markers M31 and M32. In the initial state, the measuring markers M31 and M32 are positioned in the intersecting point of the straight line with the maximum width diameter of the tumor T4 of the subject and the outline C3. Since the measuring markers M31 and M32 include the maximum width diameter of the tumor T4, the measuring markers can be positioned in the intersecting point of each straight line and the extension line of the outline C3. The outline C3 is generated to match the outline of the halo T42. The correction of the measuring markers M21, M22, M23, M24, M31, and M32 using the predetermined region or the brightness gradient information is similar to the correction of the measuring marker M11 using the region AR1 shown in FIG. 6 or the brightness gradient information.

Then, as the tumor diameter information, the measuring unit 16 obtains the width diameter and the depth diameter of the tumor based on the position information of the measuring marker of the maximum diameter surface and the width diameter based on the position information of the measuring marker of the cross section orthogonal to the maximum diameter surface (step S43).

Then, the measuring unit 16 uses the obtained width diameter a and the depth diameter c of the maximum diameter surface and the width diameter b of the cross section orthogonal to the maximum diameter surface to calculate the tumor diameter with the formula (2). The width diameter a and the depth diameter c of the maximum diameter surface of the tumor, the width diameter b of the cross section orthogonal to the maximum diameter surface, and the calculated tumor diameter are stored in the storage 17. The display combiner 18 displays the obtained width diameter a and the depth diameter c of the maximum diameter surface of the tumor, the width diameter b of the cross section orthogonal to the maximum diameter surface and the calculated tumor diameter on the display 19 (step S44). With this, the tumor diameter measuring processing ends.

As described above, according to the present embodiment, the ultrasound image diagnostic apparatus 100 includes a transmitter 12 which generates the driving signal to be output to the ultrasound probe 2, the receiver 13 which generates the acoustic ray data based on the receiving signal generated by the ultrasound probe 2, the image generator 14 which generates the B mode image data of the object from the generated acoustic ray data, the operation input unit 11 which receives input of the initial condition information for extracting the outline, the outline extractor 15 which extracts the outline of the tumor from the generated B mode image data using the input initial condition information, and the measuring unit 16 which obtains the diameter information of the tumor based on the extracted outline and calculates the feature amount of the tumor from the diameter information.

Therefore, when the feature amount of the tumor is measured, the outline is automatically extracted and the feature amount of the tumor is calculated by only inputting the initial condition information. Therefore, the burden of operation can be reduced. The feature amount of the tumor is calculated from the diameter information based on the outline extracted accurately based on the initial condition information. Therefore, the accuracy of measuring the feature amount can be enhanced. With this, the objectivity of the feature amount can be enhanced.

In the tumor depth/width ratio measuring mode, the outline extractor 15 uses the input initial condition information to extract the outline (the boundary in which the brightness difference with the surroundings is large compared to when the halo is included) of the tumor not including the halo from the generated B mode image data. Based on the extracted outline, the measuring unit 16 obtains the width diameter and the depth diameter of the tumor and calculates the depth/width ratio of the tumor from the width diameter and the depth diameter. Therefore, when the depth/width ratio of the tumor is measured, the burden of operation can be reduced, the measuring time can be shortened and the accuracy of the depth/width ratio of the tumor can be enhanced.

The outline extractor 15 calculates the outline of the tumor with the dynamic outline method based on the initial condition information and the B mode image data. Therefore, the outline in the tumor depth/width ratio measuring mode can be extracted automatically and accurately by the dynamic outline method.

In the tumor diameter measuring mode, the receiver 13 generates acoustic ray data according to the receiving signal generated by the ultrasound probe 2 in which the ultrasound is transmitted and received in the position corresponding to the maximum diameter surface of the tumor of the object and the cross section orthogonal to the maximum diameter surface. The outline extractor 15 uses the input initial condition information to extract the outline of the tumor including the halo from the generated B mode image data of the maximum diameter surface of the tumor and the B mode image data of the cross section orthogonal to the maximum diameter surface (the boundary in which the brightness difference with the surroundings is smaller than the brightness difference with the surroundings of the outline of the tumor not including the halo but which can be confirmed by sight). The measuring unit 16 obtains the width diameter and the depth diameter of the tumor based on the extracted outline corresponding to the maximum diameter surface, obtains the width diameter of the tumor based on the extracted outline corresponding to the cross section orthogonal to the maximum diameter surface, and calculates the tumor diameter from the width diameter and the depth diameter corresponding to the maximum diameter surface and the width diameter corresponding to the cross section orthogonal to the maximum diameter surface. Therefore, when the tumor diameter is measured, the burden of operation can be reduced, the measurement time can be shortened, and the accuracy of the tumor diameter can be enhanced.

The outline extractor 15 calculates the outline of the tumor of the maximum diameter surface and the outline of the tumor of the cross section orthogonal to the maximum diameter surface by the dynamic outline method based on the initial condition information and the B mode image data. Therefore, according to the dynamic outline method, the outline can be automatically and accurately extracted in the tumor diameter measuring mode.

The initial condition information is position information of the point specified from the operation input unit 11 for setting the initial outline in the dynamic outline method or the position information of the end point of the rectangle or the straight line. Therefore, the position information of one or two points needs to be input only once to generate the initial outline for extracting the outline, and the burden of operation can be reduced.

When the graph cut method is used, the initial condition information is position information of the point specified from the operation input unit 11 for setting the specified region in the graph cut method or the position information of the end point of the rectangle or the straight line. Therefore, the position information of one or two points needs to be input only once to generate the specified region for extracting the outline, and the burden of operation can be reduced even more.

The operation input unit 11 receives the operation of the correction information of the measuring marker of the extracted outline, and the measuring unit 16 obtains the diameter information of the tumor based on the measuring marker corrected by the input correction information, and calculates the feature amount of the tumor from the diameter information. Therefore, the outline of the tumor can be modified freely and the accuracy of the feature amount of the tumor can be enhanced.

The ultrasound image diagnostic apparatus 100 includes the display combiner 18. The display combiner 18 sets the predetermined region AR1 with the measuring marker in the initial state as the center, and generates the combined image data including the region AR1 and the moving measuring marker based on the correction information of the movement of the position of the measuring marker of the outline input from the operation input unit 11 to display the live image on the display 19. When the moving measuring marker is within the region AR1, the movement amount of the measuring marker for each unit of time of the operation input unit 11 is made smaller compared to when the measuring marker is outside the region AR1, and the moving measuring marker is displayed. Therefore, the measuring marker can be accurately corrected, and the correction time can be shortened.

The display combiner 18 generates the B mode image data including the moving measuring marker based on the correction information of the movement of the position of the measuring marker of the outline input from the operation input unit 11, and displays the live image on the display 19. When the brightness gradient information of the B mode image data in the position of the moving measuring marker is a predetermined threshold value or more, the movement amount of the measuring marker for each unit of time of the operation input unit 11 is made smaller compared to when the brightness gradient information is smaller than the predetermined threshold value, and the moving measuring marker is displayed. Therefore, the measuring marker can be accurately corrected and the correction time can be shortened.

The ultrasound image diagnostic apparatus 100 includes the display combiner 18 which displays the calculated feature amount of the tumor on the display 19. Therefore, the user is able to easily acknowledge the feature amount of the tumor by sight.

### Second Embodiment

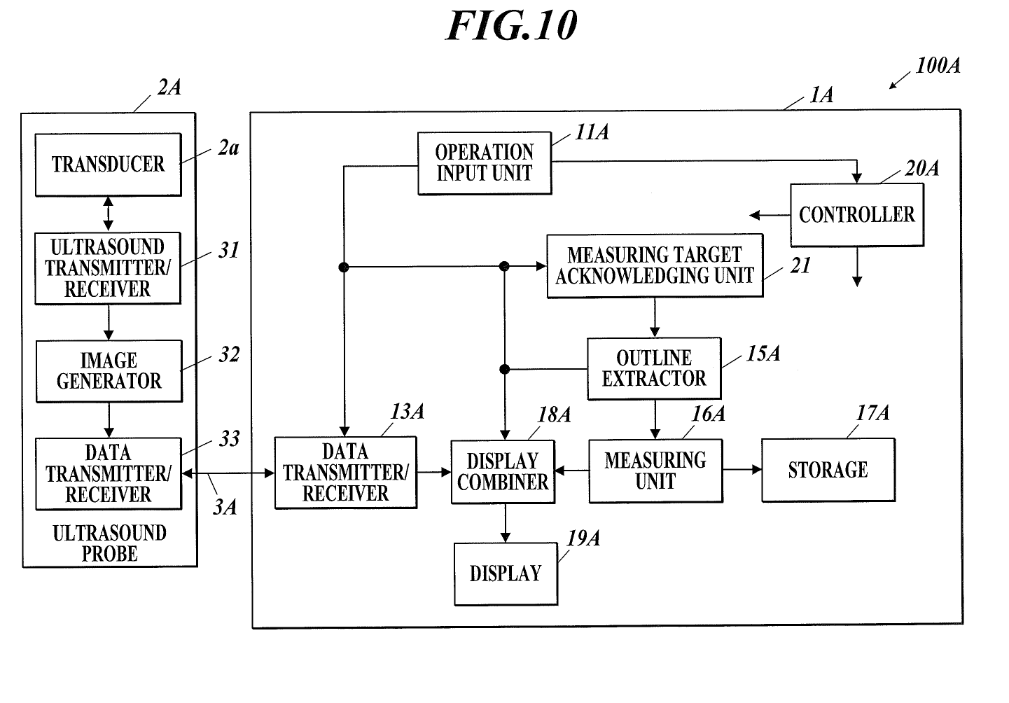

The second embodiment of the present invention is described with reference to FIG. 10 to FIG. 15. First, the configuration of the apparatus of the present embodiment is described with reference to FIG. 10. The same reference numerals are applied to the portions similar to the configuration of the apparatus of the first embodiment and the description is omitted. FIG. 10 is a block diagram showing a functional configuration of the ultrasound image diagnostic apparatus 100A of the present embodiment.

The ultrasound image diagnostic apparatus 100 of the first embodiment measures the feature amount (depth/width ratio, tumor diameter) of the tumor as the measuring target, whereas the ultrasound image diagnostic apparatus 100A of the present embodiment describes measuring the volume as the feature amount of the urinary bladder as one of the organs as the measuring object. The ultrasound image diagnostic apparatus 100A is not an apparatus similar to the ultrasound image diagnostic apparatus 100 of the first embodiment, and is an ultrasound image diagnostic system using a general purpose portable terminal. As shown in FIG. 1, the ultrasound image diagnostic apparatus 100A includes an ultrasound image diagnostic apparatus main body 1A and an ultrasound probe 2A. The configuration is not limited to the above, and for example, the ultrasound image diagnostic apparatus 100A can be an ultrasound image diagnostic apparatus including the ultrasound probe and the ultrasound image diagnostic apparatus main body, similar to FIG. 1.

The ultrasound image diagnostic apparatus main body 1A is a general purpose portable terminal and according to the present embodiment, for example, a tablet PC (personal computer) is employed. The ultrasound probe 2A includes a function to generate ultrasound image data in addition to a function to transmit and receive ultrasound. The ultrasound image diagnostic apparatus main body 1A and the ultrasound probe 2A are connected through a cable 3A. The communication method of the cable 3A is to be, for example, a USB (Universal Serial Bus), but the method is not limited to the above.

The ultrasound probe 2A includes a transducer 2a, an ultrasound transmitter/receiver 31, an image generator 32, and a data transmitter/receiver 33. The ultrasound image diagnostic apparatus main body 1A includes an operation input unit 11A, a data transmitter/receiver 13A, a measuring target acknowledging unit 21 as an initial condition obtaining unit, an outline extractor 15A, a measuring unit 16A, a storage 17A, a display combiner 18A as a third and fourth display controller, a display 19A, and a controller 20A.

Similar to the transmitter 12 and the receiver 13 of the first embodiment, the ultrasound transmitter/receiver 31 is a circuit which generates a driving signal to be supplied to the transducer 2a and receives the receiving signal based on the reflected ultrasound from the transducer 2a to generate the acoustic ray data based on the receiving signal under the control of the controller 20A. The control signal of the controller 20A is input to the ultrasound transmitter/receiver 31 and the image generator 32 through the data transmitter/receiver 13A, the cable 3A, and the data transmitter/receiver 33. According to the present embodiment, the plurality of transducers 2a of the ultrasound probe 2A are arranged in an array for a convex scanning method, but the scanning method is not limited to the above.

Similar to the image generator 14, the image generator 32 is a circuit which generates B mode image data from the acoustic ray data input from the ultrasound transmitter/receiver 31 under the control of the controller 20A. The data transmitter/receiver 33 is, for example, a communicating unit with a USB communication method, and transmits and receives data between the data transmitter/receiver 13A through the cable 3A. For example, the data transmitter/receiver 33 receives the control signal from the controller 20A from the data transmitter/receiver 13A, and outputs the above to the ultrasound transmitter/receiver 31 and the image generator 32. The data transmitter/receiver 33 receives the B mode image data input from the image generator 32 and transmits the above to the data transmitter/receiver 13A.

The operation input unit 11A is a touch panel provided as one with the display screen of the display 19A, receives input by touch from the user (tester) and outputs the operation information to the controller 20A. The data transmitter/receiver 13A is a communicating unit with a communication method such as a USB, and transmits and receives data with the data transmitter/receiver 33 through the cable 3A. For example, the data transmitter/receiver 13A transmits the control signal input from the controller 20A to the data transmitter/receiver 33. The data transmitter/receiver 13A receives the B mode image data, etc. from the data transmitter/receiver 33, and outputs the data, etc. to the measuring target acknowledging unit 21 and the display combiner 18A.

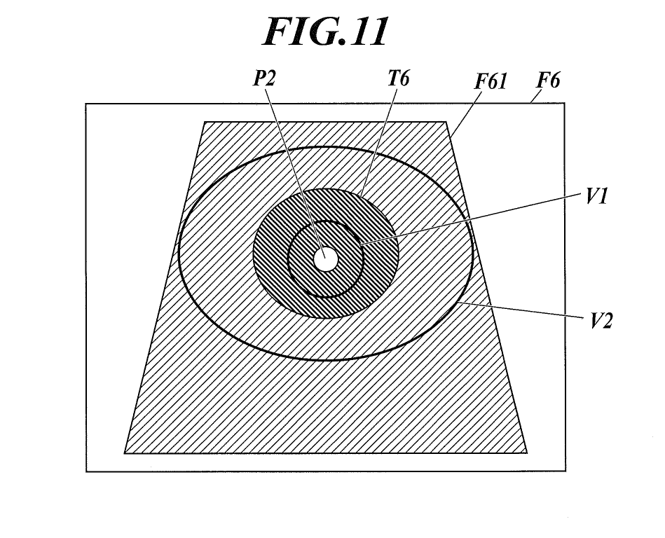

The measuring target acknowledging unit 21 is a processor. Under control of the controller 20A, the measuring target acknowledging unit 21 acknowledges whether the bladder as the measuring object has not changed for a predetermined amount of time from the live B mode image data input from the data transmitter/receiver 13A. When the measuring target acknowledging unit 21 acknowledges the above, the measuring target acknowledging unit 21 obtains the initial condition information for extracting the outline from the B mode image data, generates the measuring start information to advance to the measuring performing mode to output the information to the display combiner 18A, and outputs the B mode image data and the obtained initial condition information to the outline extractor 15A. The measuring performing mode is a mode which performs the actual measuring of the feature amount of the measuring target. Here, the method of acknowledging the measuring target by the measuring target acknowledging unit 21 is described with reference to FIG. 11. FIG. 11 is a schematic diagram showing the display image F6 including the B mode image F61 including the bladder T6.

The measuring target acknowledging unit 21 compares the plurality of B mode image data drawn in a predetermined amount of time in a unit of 2 frames. Specifically, a difference value of all pixels between two frames of the B mode image data is calculated, and it is determined whether the difference value of all pixels is a predetermined threshold value or less. When the value is a predetermined threshold value or less, it is acknowledged that there is no difference between the drawn images. When a state in which there is no difference between the images continues for a predetermined amount of time or more, it is automatically determined that the measuring target is stably drawn for a predetermined amount of time. As methods to determine the difference value of all pixels between frames include SSD (Sum of Squared Difference) method, SAD (Sum of Absolute Difference) method, etc. The SSD method is a method to calculate the sum of two times the difference between the brightness values of each pixel in the same position in the image data of two frames as the difference value of all pixels. The SAD method is a method to calculate the sum of the absolute value of the difference between the brightness values of each pixel in the same position in the image data of two frames as the difference. Here, when there is operation to change the gain, if difference calculation is corrected using the gain value, it is possible to cope with change in lighting.

When the measuring target acknowledging unit 21 acknowledges there is no change in the measuring target, the measuring target acknowledging unit 21 generates the measuring start information in the measuring performing mode to output the information to the display combiner 18A, and obtains the initial condition information from the B mode image data input from the data transmitter/receiver 13A after the timing of starting the measuring. For example, foreground V1 and background V2 of the bladder T6 are automatically obtained as the initial condition information from the image data of the B mode image F61 including the bladder T6 as the measuring target as shown in FIG. 11. The foreground V1 is a pixel with a brightness value in the B mode image F61 being smaller than a predetermined value, and is on a circumference of a circle or an oval with a predetermined radius including the pixel near the center of the image (reference point P2) as the center. Here, the organ filled with liquid inside such as the bladder, etc. is drawn with low echo or no echo compared to the surrounding tissue in the B mode image, and the brightness becomes small. Therefore, the pixel near the center of the image with the small brightness value is to be the reference point P2. The background V2 is a pixel on a circumference of a circle or an oval with a size within the end of the image with the center point of the foreground V1 as the center.

Similar to the outline extractor 15 of the first embodiment, under the control of the controller 20A, the outline extractor 15A is a processor which extracts the outline of the bladder of the measuring target in the B mode image data by the graph cut method based on the B mode image data and the initial condition information input from the measuring target acknowledging unit 21, and outputs the outline data of the extracted outline to the measuring unit 16A. Specifically, the outline extractor 15A uses the brightness of the foreground and the background of the bladder as the initial condition information input from the measuring target acknowledging unit 21 to extract the outline of the bladder by the graph cut method. For example, the average brightness of the region of the foreground in the initial condition information is used as the brightness of the bladder in the graph cut method, and the average brightness of the region of the background in the initial condition information is used as the brightness of the background of the bladder in the graph cut method. The outline extractor 15A may use the circle or the oval of the foreground or the background of the bladder in the initial condition information input from the measuring target acknowledging unit 21 as the initial outline, and extract the outline of the bladder with the Snakes method as the dynamic outline method.

Similar to the measuring unit 16 of the first embodiment, under the control of the controller 20A, the measuring unit 16A is a processor which calculates diameter information of the bladder as the measuring target, and a feature amount based on the diameter information from the outline data (corrected measuring marker) input from the outline extractor 15A, outputs the diameter information of the measuring target and the feature amount to the display combiner 18A, and stores the diameter information and the feature amount in the storage 17A. According to the present embodiment, the measuring unit 16A calculates the volume as the feature amount of the bladder.

Similar to the display combiner 18 of the first embodiment, under the control of the controller 20A, the display combiner 18A generates display image data by combining as is or suitably processing and combining the B mode image data input from the data transmitter/receiver 13A, the outline data, the measuring marker, the diameter information, and the feature amount, etc. input from the measuring unit 16A, and outputs the image signal of the display image data to the display 19A.

Similar to the display 19 of the first embodiment, the display 19A includes a display apparatus for a portable terminal such as a LCD, organic EL display, inorganic EL display, etc., and according to the image signal of the display image data input from the display combiner 18A, the display image is displayed on the display screen.

The controller 20A has a configuration similar to the controller 20 of the first embodiment, and centrally controls the operation of each unit of the ultrasound image diagnostic apparatus main body 1A and ultrasound probe 2A. Specifically, a first measuring target volume measuring program and a second measuring target volume measuring program are stored in the ROM of the controller 20A. Although the controller 20A controls each unit of the ultrasound image diagnostic apparatus main body 1A and the ultrasound probe 2A, the line showing control is omitted on FIG. 10.

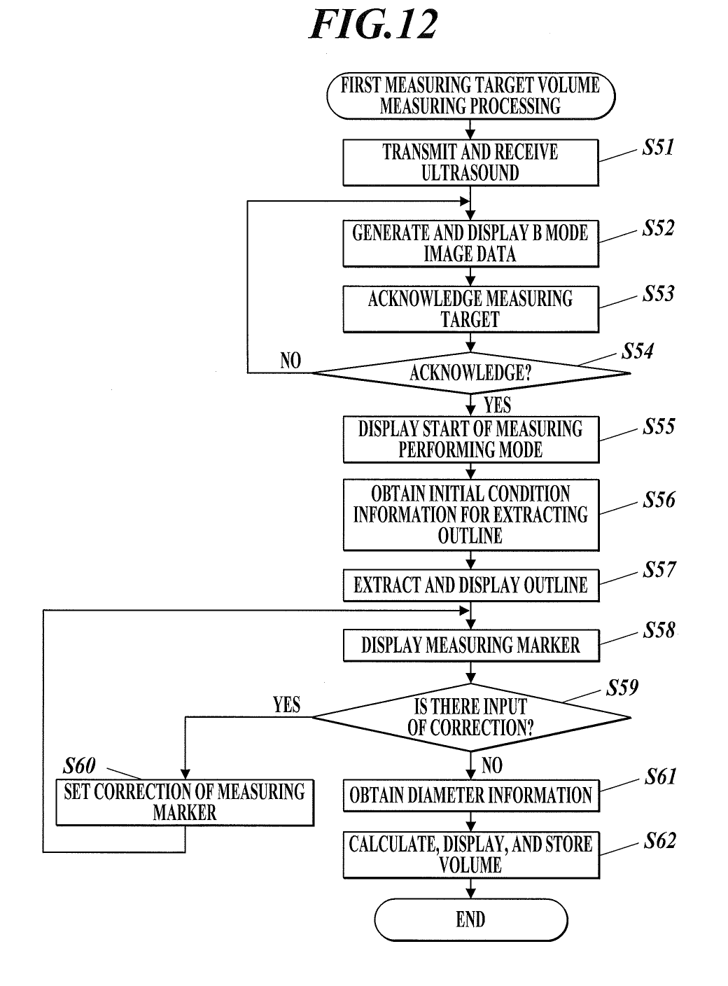

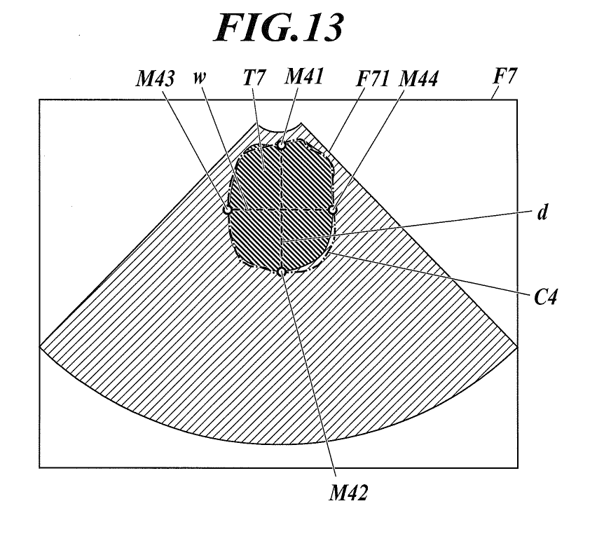

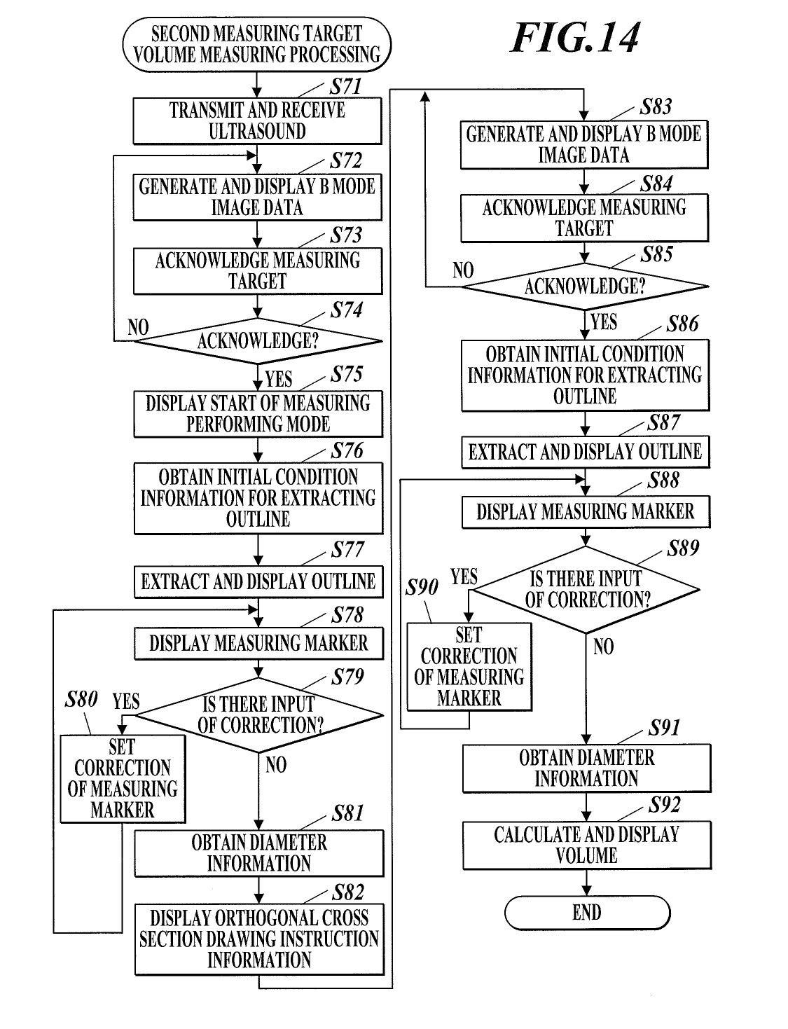

Next, the operation of the ultrasound image diagnostic apparatus 100A is described with reference to FIG. 12 to FIG. 15. FIG. 12 is a flowchart showing the first measuring target volume measuring processing. FIG. 13 is a schematic diagram showing a display image F7 including the B mode image F71 of one cross section including the bladder T7. FIG. 14 is a flowchart showing the second measuring target volume measuring processing. FIG. 15 is a schematic diagram showing a display image F8 including the B mode image F81 of the orthogonal cross section of the display image F7 shown in FIG. 13.

The first measuring target volume measuring processing performed in the ultrasound image diagnostic apparatus 100A is described with reference to FIG. 12. The first measuring target volume measuring processing automatically acknowledges that the bladder as the measuring target is stably drawn for a predetermined amount of time from the B mode image data of one cross section, and automatically calculates the outline data of the bladder, the diameter information and the volume as the feature amount. For example, when it is a specific preset point of time or when ON/OFF of automatic measurement is set in advance and it is ON, the controller 20 controls each unit to perform the first measuring target volume measuring processing according to the first measuring target volume measuring program stored in the ROM.

In the ultrasound image diagnostic apparatus 100A, first, similar to step S11 of the tumor depth/width ratio measuring processing shown in FIG. 4, the ultrasound transmitter/receiver 31 starts the transmitting and the receiving of the ultrasound for the B mode image for each unit of frames (step S51). Then, similar to step S12 of FIG. 4, the image generator 32, the data transmitter/receivers 33, 13A, and the display combiner 18A generate B mode image data and display the image based on the receiving signal of the received ultrasound (step S52). For example, the user holds the ultrasound probe 2 in the dominant hand and the ultrasound image diagnostic apparatus main body 1A in the other hand. The user operates the ultrasound probe 2A to be held against the object, confirms by sight the displayed B mode image while rotating the ultrasound probe 2 on the skin surface, and stops at the cross section in which the diameter of the bladder becomes largest on the B mode image.

Then, as acknowledgement of the measuring target, the measuring target acknowledging unit 21 calculates as the difference value of all pixels the sum value of the difference of each pixel between two frames of the B mode image data in a predetermined amount of time of the B mode image data generated in step S52 by the SSD method or the SAD method (step S53). Then, the measuring target recognizing unit 21 determines whether the difference value of all pixels calculated in step S53 is a predetermined threshold value or less and whether it acknowledges that the bladder as the measuring target is drawn stably for a predetermined amount of time (step S54). When it is not acknowledged that the measuring target is drawn stably (step S54; NO), the processing advances to step S52.

When it is acknowledged that the measuring target is drawn stably (step S54; YES), the measuring target acknowledging unit 21 generates the measuring start information to start (advance to) the measuring performing mode and outputs the information to the display combiner 18A, and the display combiner 18 displays the measuring start information on the display 19A (step S55). Then, the measuring target acknowledging unit 21 obtains and sets the initial condition information as the parameter for extracting the outline from the B mode image data generated at the timing after starting the measuring performing mode, and outputs the B mode image data and the set initial condition information to the outline extractor 15A (step S56).

Then, similar to step S16 shown in FIG. 4, the outline extractor 15A extracts the outline of the bladder as the measuring target from the B mode image data input in step S55 by the graph cut method using the initial condition information set in step S55 to generate outline data. The outline data is output to the display combiner 18 through the measuring unit 16. The display combiner 18 generates the combined image data including the outline on the B mode image based on the input B mode image data and the outline data, and displays the combined image based on the combined image data on the display 19 (step S57). In step S57, the outline extractor 15A uses the initial condition information set in step S55 and the parameters α, β stored in the storage 17A to extract the outline of the bladder as the measuring target from the B mode image data input in step S55 and to generate the outline data by the Snakes method.

Steps S58 to S60 are similar to steps S17 to S19 shown in FIG. 4. For example, in step S58, the display image F7 shown in FIG. 13 is displayed. The display image F7 includes the B mode image F71 scanning the bladder T7. The measuring markers M41, M42, M43, and M44 are positioned on the outline C4 of the bladder T7 extracted in step S57 in the B mode image F71. The measuring markers M41 and M42 are positioned in the end points of the maximum depth diameter (solid straight line in FIG. 13) on the outline C4. The maximum depth diameter is the maximum diameter of the outline of the bladder which is not necessarily in the vertical direction. The measuring markers M43 and M44 are positioned in the end points of the maximum width diameter (broken straight line in FIG. 13) orthogonal to the maximum depth diameter on the outline C4. In steps S59 and S60, the correction information of the measuring markers M41, M42, M43, and M44 can be input. Alternatively, in steps S58 to S60, the display combiner 18A may generate tomographic image data including the moving measuring marker based on the correction information of the movement of the position of the measuring marker in the outline input from the operation input unit 11A, display the live image on the display 19, and display and correct the plurality of measuring markers by moving the plurality of measuring makers in the same direction or an enlarging/reducing direction according to input of the correction information of one of the measuring markers.

When there is no input of the correction information (step S59; NO), the measuring unit 16A obtains the diameter information of the bladder from the present position information of the measuring marker (step S61). In step S61, for example, the measuring unit 16A calculates and obtains the maximum depth diameter d between the measuring markers M41 and M42 and the maximum width diameter w between the measuring markers M43 and M44 as diameter information.

Then, the measuring unit 16A calculates the volume of the bladder by using the diameter information obtained in step S61, stores the diameter information of the bladder and the calculated volume of the bladder in the storage 17A, and outputs the diameter information and the volume of the bladder to the display combiner 18A. The display combiner 18A displays the obtained diameter information of the bladder and the calculated volume of the bladder on the display 19A (step S62) and ends the first measuring target volume measuring processing. In step S62, for example, in the measuring unit 16A, the maximum depth diameter d and the maximum width diameter w obtained in step S61 are used to calculate the volume v of the bladder with the following formula (8) or the following formula (9).

V=d×w×d×π/6 (8)

V−d×w×w×π/6 (9)

In step S62, the user may freely choose either to use formula (8) or formula (9), or the maximum depth diameter d and the maximum width diameter w may be substituted in the formula (8) and formula (9) to use the formula in which the value of the volume v is larger.

Next, as shown in FIG. 14, the second measuring target volume measuring processing performed in the ultrasound image diagnostic apparatus 100A is described. The second measuring target volume measuring processing automatically acknowledges the bladder as the measuring target is stably drawn for a predetermined amount of time from the B mode image data of two cross sections, a cross section with the largest diameter (maximum diameter surface) and the B mode image data of a cross section orthogonal to the maximum diameter surface (orthogonal cross section). Then, the processing automatically calculates the outline data of the bladder, the diameter information and the volume as the feature amount. For example, when the user inputs the instruction to perform the second measuring target volume measuring processing through the operation input unit 11A, this acts as a trigger and the controller 20A controls each unit to perform the second measuring target volume measuring processing according to the second measuring target volume measuring program stored in the ROM.

Steps S71 to S81 are similar to steps S51 to S61 shown in FIG. 12. For example, in step S78, as shown in FIG. 13, a display image F7 including a B mode image F71 including the bladder T7 with the maximum diameter surface is displayed, and the outline C4 of the bladder T7 and the measuring markers M41, M42, M43, and M44 are displayed. In step S81, for example, the maximum depth diameter d between the measuring markers M41 and M42 and the maximum width diameter w between the measuring markers M43 and M44 are obtained as the diameter information of the bladder T7.

Then, the controller 20A generates the orthogonal cross section drawing instruction information to draw the orthogonal cross section orthogonal to the bladder as the measuring target for which the diameter information is obtained in step S81 and outputs the information to the display combiner 18A. The display combiner 18A displays the input orthogonal cross section drawing instruction information on the display 19 (step S82). The user confirms the B mode image by sight again, and rotates the ultrasound probe 2 on the skin surface to match to the position of the orthogonal cross section orthogonal to the maximum diameter surface.

Steps S83 to S91 are similar to the steps S72 to S74 and S76 to S81 when the drawing target is the orthogonal cross section. For example, in step S88, as shown in FIG. 15, a display image F8 including a B mode image F81 including the bladder T7 of the orthogonal cross section in the display image F7 shown in FIG. 13 is displayed, and an outline C5 of the bladder T7 and measuring markers M51 and M52 are displayed. The measuring markers M51 and M52 are positioned in the two end points of the maximum diameter on the outline C5. For example, in step S91, the maximum diameter h between the measuring markers M51 and M52 is calculated and obtained.

Then, the measuring unit 16A calculates the volume of the bladder by using the diameter information obtained in steps S81, S91, stores the diameter information of the bladder and the calculated volume of the bladder in the storage 17A, and outputs the diameter information of the bladder and the volume to the display combiner 18A. The display combiner 18A displays the obtained diameter information of the bladder and the calculated diameter information of the bladder and displays the above on the display 19A (step S92). With this, the second measuring target volume measuring processing ends. In step S92, for example, the measuring unit 16A uses the maximum depth diameter d and the maximum width diameter w obtained in step S81 and the maximum diameter h obtained in step S91 to calculate the volume v with the following formula (10).

v=d×w×h×π/6 (10)

As described above, according to the present embodiment, the ultrasound image diagnostic apparatus 100A includes an ultrasound transmitter/receiver 31 which supplies the driving signal to the transducer 2a of the ultrasound probe 2A, to generate the acoustic ray data based on the receiving signal received from the transducer 2a, the image generator 32 which generates the B mode image data from the generated acoustic ray data, the measuring target acknowledging unit 21 which obtains the initial condition information for extracting the outline of the bladder, an outline extractor 15A which uses the obtained initial condition information to extract the outline of the bladder from the generated B mode image data, and a measuring unit 16A obtains the diameter information of the bladder based on the extracted outline and calculates the feature amount of the bladder from the diameter information.

Therefore, when the feature amount of the bladder is measured, the initial condition information is obtained, the outline is automatically extracted, and the feature amount of the bladder is calculated. Therefore, the burden of operation can be reduced. The feature amount of the bladder is calculated from the diameter information based on the outline accurately extracted based on the initial condition information. Therefore, the accuracy of measuring the feature amount and the objectivity of the feature amount can be enhanced. Specifically, since the ultrasound image diagnostic apparatus main body 1A is a portable terminal, even if the user holds the ultrasound image diagnostic apparatus main body 1A and the ultrasound probe 2A in each hand and both hands are full, the burden of operation reduces and the feature amount of the bladder can be easily measured.

The ultrasound image diagnostic apparatus 100A includes a measuring target acknowledging unit 21 which acknowledges that the bladder is drawn stably for a predetermined amount of time from the generated B mode image data and when it is acknowledged, the processing advances to the measuring performing mode, and when the processing is advanced, the initial condition information for extracting the outline is obtained. Therefore, the measuring can be started automatically, and the burden of operation can be further reduced.

The measuring target acknowledging unit 21 obtains the initial condition information for extracting the outline from the generated B mode image data. Therefore, the initial condition information can be automatically obtained and the burden of operation can be further reduced.

The outline extractor 15A uses the obtained initial condition information to extract the outline of the maximum diameter surface of the measuring target from the generated B mode image data. Based on the extracted outline, the measuring unit 16A obtains the maximum depth diameter and the maximum width diameter orthogonal to the maximum depth diameter as the diameter information of the bladder, and calculates the volume as the feature amount of the bladder from the maximum depth diameter and the maximum width diameter. Therefore, when the volume of the bladder is measured, the burden of operation can be reduced, the measuring time can be shortened, and the accuracy of volume of the bladder can be enhanced. Since the volume of the bladder is calculated using the diameter information, compared to calculating the volume of the bladder using square area, the processing speed to calculate the volume of the bladder can be made fast. The volume of the bladder may be calculated using the square area and the diameter information of the bladder. According to such configuration, the accuracy of the volume of the bladder can be enhanced.

The ultrasound transmitter/receiver 31 generates the acoustic ray data based on the receiving signal generated by the transducer with which the ultrasound is transmitted and received in the position corresponding to the maximum diameter surface of the measuring target of the object and the cross section orthogonal to the maximum diameter surface. The outline extractor 15A uses the obtained initial condition information to extract the outline of the bladder from the generated B mode image data of the maximum diameter surface, and extracts the outline of the bladder from the B mode image data of the cross section orthogonal to the maximum diameter surface. The measuring unit 16A obtains the maximum depth diameter and the maximum width diameter orthogonal to the maximum depth diameter as the diameter information of the bladder based on the extracted outline corresponding to the maximum diameter surface, obtains the maximum diameter as the diameter information of the bladder based on the extracted outline corresponding to the cross section orthogonal to the maximum diameter surface, and calculates the volume as the feature amount of the bladder from the maximum depth diameter, the maximum width diameter, and the maximum diameter. Therefore, when the volume of the bladder is measured, by using two cross sections, the accuracy of the volume of the bladder can be further enhanced.

The outline extractor 15A calculates the outline of the bladder by the graph cut method based on the initial condition information and the B mode image data. Therefore, according to the graph cut method, the outline when the volume of the bladder is measured can be automatically and accurately extracted.

When the graph cut method is used, the initial condition information is the brightness information of the foreground and the background. By automatically obtaining the above, there is no need to input information to generate the specifying region for extracting the outline, and the burden of operation can be reduced even more.

The outline of the bladder can be extracted using the Snakes method. In this case, the initial condition information is the initial outline in the Snakes method. By automatically obtaining the above, there is no need to input information to generate the initial outline for extracting the outline, and the burden of operation can be reduced even more.

The display combiner 18A may generate the tomographic image data including the moving measuring marker based on the correction information of the movement of the position of the measuring marker in the outline input from the operation input unit 11A and display the live image on the display 19A. The plurality of measuring markers may be moved together in the same direction or the enlarging/reducing direction and displayed and corrected according to the input of the correction information of one measuring marker. Therefore, all measuring markers can be similarly corrected in response to the tendency of extracting the outline.

The display combiner 18A displays the display information showing the start of (advance to) the measuring performing mode on the display 19A when the process advances to the measuring performing mode. Therefore, the user is able to easily confirm the switch to the measuring performing mode by sight.

The measuring target acknowledging unit 21 calculates the difference of the entire frame of the plurality of frames of the generated B mode image data and when the calculated difference value is a predetermined threshold value or less, the process advances to the measuring performing mode. Therefore, the tester can move the ultrasound probe 2A, capture the bladder of the measuring target and stop, and detect accurately and automatically that the preparation is complete to start the measuring performing mode. Therefore, it is possible to omit the user's burden of inputting the freeze button, and the burden of operation can be reduced even more.

### (Modification)

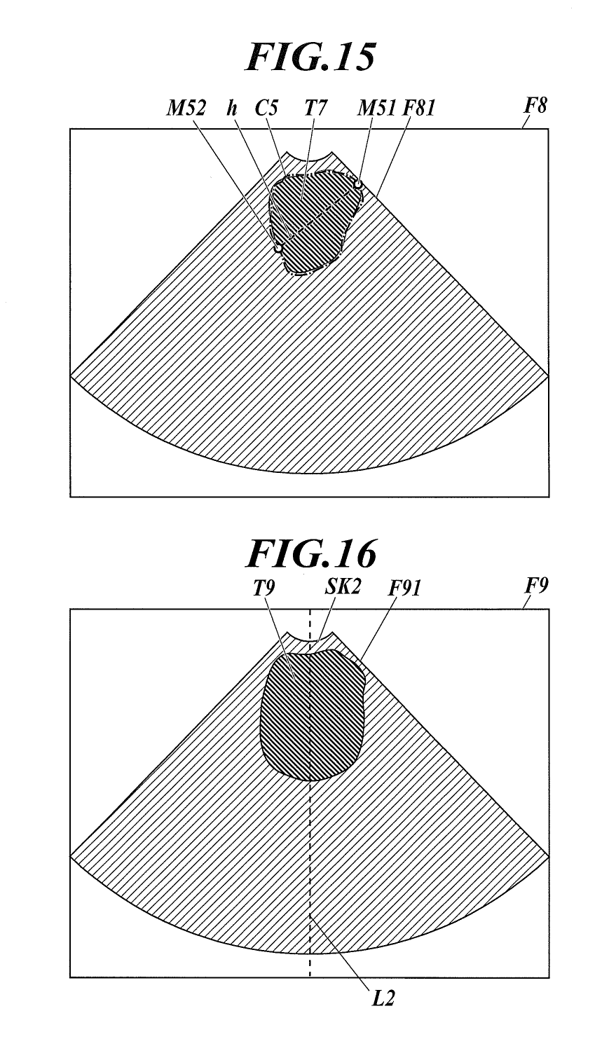

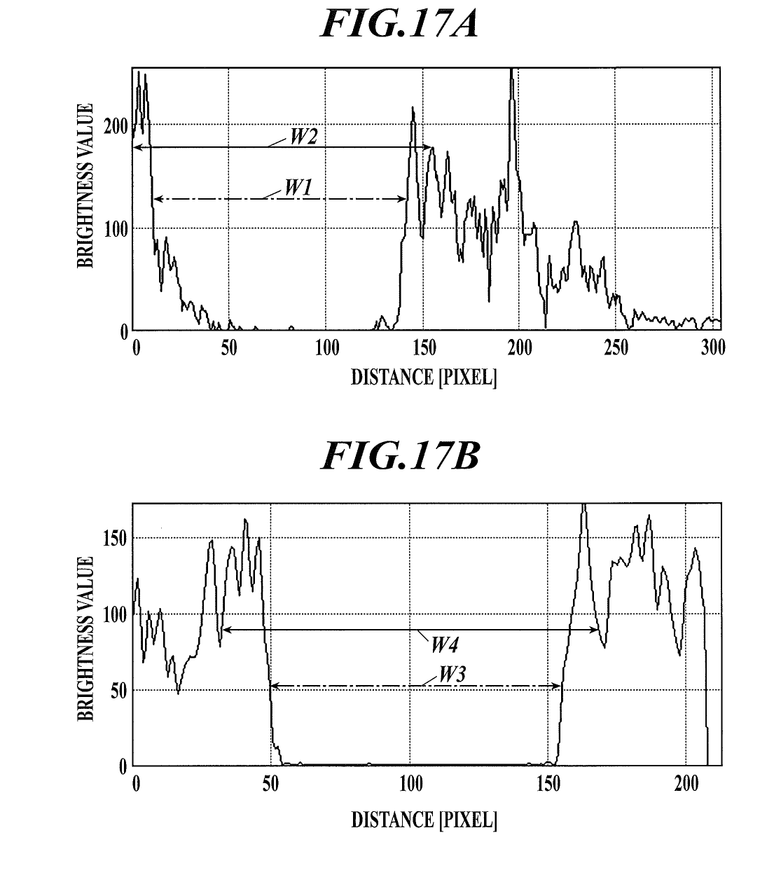

The modification of the second embodiment is described with reference to FIG. 16 to FIG. 17B. According to the above modification, the ultrasound image diagnostic apparatus 100A of the second embodiment is used, and the first or second measuring target volume measuring processing is similarly performed. The processing that the measuring target acknowledging unit 21 acknowledges the measuring target is drawn stably for a predetermined amount of time is different. Only the different processing is described, and the description of other apparatus configuration and processing is omitted.

The processing of the measuring target acknowledging unit 21 of the modification is described with reference to FIG. 16, FIG. 17A, and FIG. 17B. FIG. 16 is a schematic diagram showing a display image F9 including the B mode image F91 including a bladder T9. FIG. 17A is a diagram showing a brightness profile in a reference line of a cross section in a vertical direction. FIG. 17B is a diagram showing a brightness profile in a reference line in a horizontal direction from a reference point of a reference line in a vertical direction shown in FIG. 17A.

According to the modification, under the control of the controller 20A, the measuring target acknowledging unit 21 acknowledges from the live B mode image data input from the data transmitter/receiver 13A that the bladder as the measuring target does not change for a predetermined amount of time. When the measuring target acknowledging unit 21 acknowledges the above, the measuring target acknowledging unit 21 obtains the initial condition to extract the outline, generates the measuring start information to advance to the measuring performing mode to output the information to the display combiner 18A, and outputs the B mode image data and the obtained initial condition information to the outline extractor 15A.

Here, processing to acknowledge stable drawing of the bladder as the measuring target for a predetermined amount of time is described. First, the measuring target acknowledging unit 21 sets a reference line on the B mode image including the bladder drawn in a predetermined amount of time. The reference line is set vertical to the skin surface and in a vertical direction from the center of the left and right direction (horizontal direction) of the B mode image. For example, as shown in FIG. 16, a reference line L2 is set from the input B mode image F91 (of the display image F9) including the bladder T9 vertical to the skin surface SK2 and in a vertical direction from the center of the left and right direction of the B mode image F71. The reference line L2 is a line corresponding to an acoustic line at a center of the B mode image in the frame of the B mode image data. Then, the measuring target acknowledging unit 21 generates the brightness profile in the reference line in the vertical direction for two frames in the B mode image data drawn in a predetermined amount of time.

As shown in FIG. 17A, the brightness profile is generated by plotting a brightness value in the reference line with a distance from the skin surface in the horizontal axis (pixel number) and a brightness value in the vertical axis (gray value). Then, the measuring target acknowledging unit 21 determines whether the value and the shape (brightness value high low high) changes in the brightness profile for the reference line in the two frames in the predetermined amount of time. When the change in the value and the shape of the brightness profile for the reference line is a threshold value or less, the measuring target acknowledging unit 21 automatically determines that there is no change in the measuring target and that there is no difference in the measuring image, and therefore, the measuring target is drawn stably in a predetermined amount of time.

When the measuring target acknowledging unit 21 acknowledges the measuring target, the measuring target acknowledging unit 21 generates the measuring start information of the measuring performing mode to output the information to the display combiner 18A and obtains the initial condition information from the B mode image data input from the data transmitter/receiver 13A after the timing of starting the measuring. For example, the measuring target acknowledging unit 21 generates the brightness profile for the reference line in the vertical direction in the B mode image data input after the timing of starting the measuring (for example, brightness profile shown in FIG. 17A). The center of the width W1 (for example, both arrows of the short and long dash line in FIG. 17A) of the low brightness region with the predetermined low brightness is to be the reference point. The pixel on the circumference of the circle or oval including the predetermined radius is to be the foreground as the initial condition information. The pixel on the circumference of the circle or the oval with the diameter including the width W2 (for example, both arrows of the solid line in FIG. 17A) of the high brightness region with the low brightness region in between is to be the background as the initial condition information.

Alternatively, the measuring target acknowledging unit 21 generates the brightness profile (for example, brightness profile shown in FIG. 17A) for the reference line in the vertical direction of the B mode image data input after the timing of starting the measuring. The center of the width W1 of the low brightness region with the predetermined low brightness is to be the reference point in the vertical direction. The predetermined radius according to the reference point in the vertical direction and the width W1 is to be the radius of the foreground in the vertical direction. The radius including the width W2 of the high brightness portion with the low brightness in between is to be the radius of the background in the vertical direction. The measuring target acknowledging unit 21 sets the reference line in the horizontal direction from the set reference point in the vertical direction, and generates the brightness profile for the reference line in the horizontal direction (for example, brightness profile shown in FIG. 17B). The center of the width W3 (for example, both arrows of the short and long dash line shown in FIG. 17B) of the low brightness region with the predetermined low brightness based on the brightness profile in the horizontal direction is to be the reference point in the horizontal direction. The predetermined radius according to the reference point in the horizontal direction and the width W3 is to be the radius of the foreground in the horizontal direction. The radius including the width W4 (for example, both arrows of the solid line in FIG. 17B) in the high brightness region with the low brightness region in between is to be the radius of the background in the horizontal direction. The circle or the oval including the radius in the vertical direction and the radius in the horizontal direction is to be the foreground and the background as the initial condition information. As described above, when the foreground and the background of the initial condition information is generated using not only the brightness profile for the reference line in the vertical direction but also the brightness profile for the reference line in the horizontal direction, the accuracy of extracting the outline of the bladder as the measuring target is enhanced.

The measuring target acknowledging unit 21 performs the process of acknowledging the measuring target of the above-described modification and obtaining the initial condition information in step S53 of the first measuring target volume measuring processing shown in FIG. 12 and steps S73, S84 of the second measuring target volume measuring processing shown in FIG. 13.

According to the above-described modification, the measuring target acknowledging unit 21 generates the brightness profile for the predetermined line in the generated B mode image data and uses the generated brightness profile to obtain the initial condition information based on the foreground and the background of the graph cut method. Therefore, the suitable initial condition information can be easily obtained and the outline of the bladder can be extracted easily and accurately.

The measuring target acknowledging unit 21 calculates the difference of the reference line L2 corresponding to the one acoustic ray in the center of the B mode image in the plurality of frames of the generated B mode image data. When the calculated difference value is a predetermined threshold value or less, the processing advances to the measuring performing mode. Therefore, the calculation does not have to be performed for all of the pixels in the B mode image, and the calculating amount reduces. With this, the processing speed becomes faster. Moreover, when the B mode image is drawn with the reference line L2 displayed on the screen in advance, the user can be guided to draw the object in the suitable position.

The description of the embodiments and the modification are merely examples of the preferable ultrasound image diagnostic apparatus according to the present invention, and the present invention is not limited to the above.

For example, according to the embodiments and the modification, the extracted outline is automatically displayed on the B mode image, but the present invention is not limited to the above. Alternatively, the extracted outline may not be displayed in the B mode image.

According to the embodiments and the modification, the measuring marker is initially positioned on the outline or the extension line of the outline but the present invention is not limited to the above. The measuring marker may be positioned initially in the position separated a predetermined distance in the internal direction or the outer direction of the measuring target from a point on the outline or on the extension line of the outline. According to such configuration, the correction direction of the measuring marker can be substantially unified in a certain direction (outer direction or internal direction of the measuring target), and the operability of correcting the position of the measuring marker can be enhanced.

According to the above embodiments and the modification, the display combiner 18 as the output controller displays the diameter information of the measuring target and the feature amount on the display 19, but the present invention is not limited to the above. For example, the ultrasound image diagnostic apparatus 100 includes the communicating unit as the output controller which is able to communicate with an external apparatus or storage (not shown), the communicating unit transmits the B mode image data, and the diameter information and the feature amount of the measuring target to an output unit such as an external printing apparatus, storage apparatus, etc. and prints, stores, etc. such information.

At least two among the first embodiment, the second embodiment, and the modification can be suitably combined. For example, instead of step S13 of the tumor depth/width ratio measuring processing shown in FIG. 4, steps S33, S36 of the tumor diameter measuring processing shown in FIG. 8, and steps S53, S54 shown in FIG. 12 as described in the first embodiment, the controller 20 is configured to acknowledge that the tumor as the measuring target does not change for a predetermined amount of time, and when the controller 20 acknowledges the above, the processing advances to the measuring processing mode (and the processing continues).

Instead of step S15 of the tumor depth/width ratio measuring processing shown in FIG. 4, step S38 of the tumor diameter measuring processing shown in FIG. 8, step S56 shown in FIG. 12 as described in the first embodiment, the measuring target acknowledging unit 21 automatically obtains the initial condition information as the parameter for extracting the outline from the B mode image data. The first embodiment uses the ultrasound image diagnostic apparatus 100A including the ultrasound image diagnostic apparatus main body 1A as the portable terminal and performs the tumor depth/width ratio measuring processing and the tumor diameter measuring processing.

The tumor is the measuring target in the first embodiment and the bladder is the measuring target in the second embodiment and the modification, but the present invention is not limited to the above. The measuring target can be a lesion, organ, tissue, component, etc. of the object other than the tumor and the bladder.

According to the second embodiment and the modifications, one frame of the cross section after advancing to the measuring mode is used to extract the outline of the bladder and to calculate the diameter information, but the present invention is not limited to the above. The outline extractor 15A may store the plurality of frames (after advancing to the measuring mode) in the storage 17A, and extract the outline of the bladder in each frame. The measuring unit 16A as the selecting unit may calculate the diameter information from the outline data of each frame, automatically select the maximum diameter information among the plurality of calculated diameter information, and calculate the volume of the bladder from the selected maximum diameter information. According to such configuration, when the feature amount of the bladder is measured, the initial condition information is obtained, and automatically, the outline is extracted, the maximum diameter information is selected, and the feature amount of the bladder is calculated. Therefore, the burden of operation on the user can be reduced. The feature amount of the bladder is calculated from the maximum diameter information based on the accurately extracted outline based on the initial condition information. Therefore, the accuracy of measuring the feature amount can be enhanced, and the objectivity of the feature amount improves.

Alternatively, the outline extractor 15A may store the plurality of frames (after advancing to the measuring mode) in the storage 17A, and extract the outline of the bladder in each frame. The measuring unit 16A may calculate the diameter information from the outline data of each frame. The operation input unit 11A as the selecting unit receives one selection input from the user among the plurality of calculated diameter information of the bladder. The measuring unit 16A may calculate the volume of the bladder from the selected diameter information. According to the above configuration, when the feature amount of the bladder is calculated, the outline is clearly drawn, and the user is able to select the diameter information of the suitable frame. With this, the accuracy of measuring the feature amount is enhanced.

Alternatively, when there are two cross sections, for each cross section, the outline extractor 15A may store the plurality of frames (after advancing to the measuring mode) in the storage 17A, and extract the outline of each bladder. The measuring unit 16A may calculate the diameter information from the outline data of each frame, automatically select the maximum diameter information among the plurality of calculated diameter information, and calculate the volume of the bladder from the selected maximum diameter information. Alternatively, even when there are two cross sections, for each cross section, the outline extractor 15A stores the plurality of frames (after switching to the measuring mode) in the storage 17A, and extracts the outline of the bladder in each frame. The measuring unit 16A may calculate the diameter information from each frame of the outline data. The operation input unit 11A may receive one selection input by the user among the plurality of calculated diameter information of the bladder. The measuring unit 16A may calculate the volume of the bladder from the selected diameter information.

Alternatively, the configuration in which the diameter information of the plurality of frames is calculated, and the diameter information of one frame is selected from the plurality of calculated diameter information automatically or according to user operation, and the feature amount of the measuring target is calculated using the selected diameter information may be applied to the first embodiment.

The detailed configuration and the detailed operation of each unit in the ultrasound image diagnostic apparatuses 100, 100A according to the above embodiments can be suitably changed without leaving the scope of the present invention.

What is claimed is:

1. An ultrasound image diagnostic apparatus which transmits and receives ultrasound with an ultrasound probe which transmits to an object transmitting ultrasound in response to a driving signal and receives reflected ultrasound to generate a receiving signal, the ultrasound image diagnostic apparatus comprising:
a transmitter which supplies a driving signal to a transducer of the ultrasound probe;
a receiver which generates acoustic ray data based on a receiving signal received from the transducer;
an image generator which generates tomographic image data of the object from the generated acoustic ray data;
a measuring target acknowledging unit which acknowledges a measuring target of the object from the generated tomographic image data is stably drawn for a predetermined amount of time and when it is acknowledged, advances to a measuring performing mode of the measuring target;
an initial condition obtaining unit which obtains initial condition information for extracting an outline of the measuring target when advancing to the measuring performing mode;
an outline extractor which uses the obtained initial condition information to extract an outline of the measuring target from the generated tomographic image data; and
a measuring unit which obtains diameter information of the measuring target based on the extracted outline and calculates the feature amount of the measuring target from the diameter information.

2. The ultrasound image diagnostic apparatus of claim 1, wherein the initial condition obtaining unit receives input of the initial condition information for extracting the outline and obtains the initial condition information.

3. The ultrasound image diagnostic apparatus of claim 1, wherein the initial condition obtaining unit obtains the initial condition information for extracting the outline from the generated tomographic image data.

4. The ultrasound image diagnostic apparatus of claim 1, wherein,
the outline extractor uses the obtained initial condition information to extract a first outline of the measuring target from the generated tomographic image data; and
based on the extracted first outline, the measuring unit obtains a first diameter and a second diameter orthogonal to the first diameter as diameter information of the measuring target, and calculates a depth/width ratio of the measuring target as a feature amount of the measuring target from the first diameter and the second diameter.

5. The ultrasound image diagnostic apparatus of claim 1, wherein,
the receiver generates acoustic ray data based on a receiving signal obtained by transmitting and receiving ultrasound in a position corresponding to a maximum diameter surface of the measuring target of the object and a cross section orthogonal to the maximum diameter surface;
the outline extractor uses the obtained initial condition information to extract a second outline of the measuring target from the generated tomographic image data of the maximum diameter surface and to extract a third outline of the measuring target from the tomographic image data of a cross section orthogonal to the maximum diameter surface; and
the measuring unit obtains a third diameter and a fourth diameter orthogonal to the third diameter as diameter information of the measuring target based on the extracted second outline corresponding to the maximum diameter surface, obtains a fifth diameter as the diameter information of the measuring target based on the extracted third outline corresponding to a cross section orthogonal to the maximum diameter surface, and calculates a measuring target diameter as a feature amount of the measuring target from the third diameter, the fourth diameter and the fifth diameter.

6. The ultrasound image diagnostic apparatus of claim 1, wherein,
the outline extractor uses the obtained initial condition information to extract a fourth outline of the measuring target from the generated tomographic image data;
the measuring unit obtains a sixth diameter and a seventh diameter orthogonal to the sixth diameter as diameter information of the measuring target based on the extracted fourth outline and calculates volume as the feature amount of the measuring target from the sixth diameter and the seventh diameter.

7. The ultrasound image diagnostic apparatus of claim 1, wherein,
the receiver generates the acoustic ray data based on a receiving signal obtained by transmitting and receiving the ultrasound in a position corresponding to a maximum diameter surface of the measuring target of the object and a cross section orthogonal to the maximum diameter surface;
the outline extractor uses the obtained initial condition information to extract a fifth outline of the measuring target from the generated tomographic image data of the maximum diameter surface and to extract a sixth outline of the measuring target from the tomographic image data of the cross section orthogonal to the maximum diameter surface; and
the measuring unit obtains an eight diameter and a ninth diameter orthogonal to the eight diameter and a ninth diameter orthogonal to the eight diameter as the diameter information of the measuring target based on the extracted fifth outline corresponding to the maximum diameter surface, obtains a tenth diameter as the diameter information of the measuring target based on the extracted sixth outline corresponding to a cross section orthogonal to the maximum diameter surface, and calculates volume as the feature amount of the measuring target from the eight diameter, the ninth diameter, and the tenth diameter.

8. The ultrasound image diagnostic apparatus of claim 1, wherein, the outline extractor extracts the outline by the graph cut method based on the initial condition information and the tomographic image data.

9. The ultrasound image diagnostic apparatus of claim 8, wherein, the initial condition information is position information of a point for setting a specifying region of the graph cut method, a position information of an end point of a rectangle or a straight line, or brightness information of a foreground and a background.

10. The ultrasound image diagnostic apparatus of claim 1, wherein the outline extractor extracts the outline by a dynamic outline method based on the initial condition information and the tomographic image data.

11. The ultrasound image diagnostic apparatus of claim 10, wherein the initial condition information is position information of a point for setting the initial outline in the dynamic outline method, position information of an end point of a rectangle or a straight line, or an initial outline.

12. The ultrasound image diagnostic apparatus of claim 1, further comprising, an operation input unit which receives input of correction information of a position of a measuring marker of the extracted outline,
wherein, the measuring unit obtains diameter information of the measuring target from the measuring marker corrected by the input correction information and calculates a feature amount of the measuring target from the diameter information.

13. The ultrasound image diagnostic apparatus of claim 12, further comprising a first display controller which sets a predetermined region with the measuring marker in the initial state as the center, generates tomographic image data including the predetermined region and a moving measuring marker based on the correction information of the movement of a position of the measuring marker in the outline input from the operation input unit and displays the tomographic image data live on the display, wherein when the moving measuring marker is within the predetermined region, the moving measuring marker is displayed with the movement amount of the measuring marker made smaller for each unit of time of the operation input unit compared to when the moving measuring marker is outside the predetermined region.

14. The ultrasound image diagnostic apparatus of claim 12, further comprising a second display controller which generates tomographic image data including a moving measuring marker based on the correction information of the movement of a position of the measuring marker in the outline input from the operation input unit and displays the tomographic image data live on the display, wherein when brightness gradient information of the tomographic image data in the position of the moving measuring marker is a predetermined threshold value or more, the moving measuring marker is displayed with the movement amount of the measuring marker made smaller for each unit of time of the operation input unit compared to when the brightness gradient information is smaller than the predetermined threshold value.

15. The ultrasound image diagnostic apparatus of claim 12, further comprising a third display controller which generates tomographic image data including a moving measuring marker based on correction information of the movement of a position of the measuring marker in the outline input from the operation input unit and displays the tomographic image data live on the display, wherein display is performed moving a plurality of measuring markers together in a same direction or an enlarging/reducing direction according to input of the correction information of one measuring marker.

16. The ultrasound image diagnostic apparatus of claim 1, further comprising a fourth display controller which displays display information showing switching to the measuring performing mode on the display when the mode switches to the measuring performing mode.

17. The ultrasound image diagnostic apparatus of claim 1, further comprising an output controller which outputs to an output unit the feature amount of the calculated measuring target.

18. The ultrasound image diagnostic apparatus of claim 1, wherein the measuring target acknowledging unit calculates a difference of an entire frame or a predetermined portion in the frame of a plurality of generated tomographic image data, and switches to the measuring performing mode when the calculated difference value is a predetermined threshold value or less.

19. An ultrasound image diagnostic apparatus which transmits and receives ultrasound with an ultrasound probe which transmits to an object transmitting ultrasound in response to a driving signal and receives reflected ultrasound to generate a receiving signal, the ultrasound image diagnostic apparatus comprising:
a transmitter which supplies a driving signal to a transducer of the ultrasound probe;
a receiver which generates acoustic ray data based on a receiving signal from the transducer;
an image generator which generates tomographic image data of the object from the generated acoustic ray data;
an initial condition obtaining unit which obtains initial condition information for extracting an outline of a measuring target of the object;
an outline extractor which uses the obtained initial condition information to extract an outline of the measuring target from the generated tomographic image data; and
a measuring unit which obtains diameter information of the measuring target based on the extracted outline and calculates the feature amount of the measuring target from the diameter information.

20. An ultrasound image diagnostic apparatus which transmits and receives ultrasound with an ultrasound probe which transmits to an object transmitting ultrasound in response to a driving signal and receives reflected ultrasound to generate a receiving signal, the ultrasound image diagnostic apparatus comprising:
a transmitter which supplies a driving signal to a transducer of the ultrasound probe;
a receiver which generates acoustic ray data based on a receiving signal received from the transducer;
an image generator which generates tomographic image data of the object from the generated acoustic ray data;
an initial condition obtaining unit which obtains initial condition information for extracting an outline of a measuring target of the object;
an outline extractor which uses the obtained initial condition information to extract an outline of the measuring target from a plurality of frames of the generated tomographic image data;
a measuring unit which obtains diameter information of the measuring target based on the extracted outline of the plurality of frames; and
a selecting unit which selects diameter information of one frame among the obtained diameter information of the plurality of frames,
wherein the measuring unit calculates a feature amount of the measuring target from the selected diameter information.

21. The ultrasound image diagnostic apparatus of claim 20, wherein the selecting unit automatically selects diameter information with a maximum diameter among the obtained diameter information of the plurality of frames.

22. The ultrasound image diagnostic apparatus of claim 20, wherein the selecting unit receives input of selection of diameter information of one frame among the obtained diameter information of the plurality of frames.
